SEQUENCE LISTING (9 sequences)

SEQ ID NO: 1 - protein, 166 aa, Artificial Sequence
  synthetic construct; recombinant EPO
APPRLICDSRVLERYLLEAQEAENITTGCAEHCSLNENITVPDTQVNFYAWKRMEVGQQAVEVWQGLALLSEAVLRGQALLVNSSQPWEPLQLHVDQAVSGLRSLTTLLRALGAQKEAISPPDAASAAPLRTITADTFRKLFRVYSNFLRGKLKLYTGEACRTGDR

SEQ ID NO: 2 - protein, 563 aa, Artificial Sequence
  synthetic construct; recombinant epo
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFHHHHHHAPPRLICDSRVLERYLLEAQEAENITTGCAEHCSLNENITVPDTQVNFYAWKRMEVGQQAVEVWQGLALLSEAVLRGQALLVNSSQPWEPLQLHVDQAVSGLRSLTTLLRALGAQKEAISPPDAASAAPLRTITADTFRKLFRVYSNFLRGKLKLYTGEACRTGDR

SEQ ID NO: 3 - protein, 165 aa, Artificial Sequence
  synthetic construct; recombinant EPO
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLSLTTLLRALGAQKEAISPPDAASAAPLRTITADTFRKLFRVYSNFLRGKLKLYTGEACRTGDR

SEQ ID NO: 4 - DNA, 498 bp, Artificial Sequence
  synthetic construct; recombinant EPO
gctccgccgcgcctgatctgtgactctcgtgtcctggaacgctatctgctggaagcgcaggaagccgaaaacattaccacgggctgcgccgaacattgtagcctgaacgaaaatatcaccgttccggatacgcaggtcaatttttatgcatggaaacgtatggaagtcggccagcaagctgtggaagtttggcaaggtctggcactgctgtctgaagcagtgctgcgtggtcaggcactgctggttaacagctctcaaccgtgggaaccgctgcagctgcacgtcgaccaagccgtgagtggtctgcgttccctgaccacgctgctgcgtgcactgggtgctcagaaagaagcgatttcaccgccggatgcagcatcggcagctccgctgcgtaccatcacggcagacacctttcgtaaactgttccgcgtttactccaatttcctgcgcggtaaactgaaactgtatacgggtgaagcctgtcgcacgggtgaccgc

SEQ ID NO: 5 - DNA, 7162 bp, Artificial Sequence
  synthetic construct; recombinant EPO
ccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagccagtccgtttaggtgttttcacgagcacttcaccaacaaggaccatagattatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccatcaccatcatcaccacgctccgccgcgcctgatctgtgactctcgtgtcctggaacgctatctgctggaagcgcaggaagccgaaaacattaccacgggctgcgccgaacattgtagcctgaacgaaaatatcaccgttccggatacgcaggtcaatttttatgcatggaaacgtatggaagtcggccagcaagctgtggaagtttggcaaggtctggcactgctgtctgaagcagtgctgcgtggtcaggcactgctggttaacagctctcaaccgtgggaaccgctgcagctgcacgtcgaccaagccgtgagtggtctgcgttccctgaccacgctgctgcgtgcactgggtgctcagaaagaagcgatttcaccgccggatgcagcatcggcagctccgctgcgtaccatcacggcagacacctttcgtaaactgttccgcgtttactccaatttcctgcgcggtaaactgaaactgtatacgggtgaagcctgtcgcacgggtgaccgctgaggatcctctagagtcgacctgcaggcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcagcttggctgttttggcggatgagataagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 6 - DNA, 582 bp, homo sapiens
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 7 - protein, 367 aa, Artificial Sequence
  synthetic construct; maltose binding protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 8 - protein, 192 aa, homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLSLTTLLRALGAQKEAISPPDAASAAPLRTITADTFRKLFRVYSNFLRGKLKLYTGEACRTGDR

SEQ ID NO: 9 - protein, 6 aa, tobacco etch virus
ENLYFQ

(19) United States
(12) Patent Application Publication
(10) Pub. No.: US 2023/0151071 A1
(43) Pub. Date: May 18, 2023

(54) COMPOSITIONS COMPRISING RECOMBINANT EPO AND METHODS OF USE THEREOF

(71) Applicants: UNITED STATES GOVERNMENT AS REPRESENTED BY THE DEPARTMENT OF VETERANS AFFAIRS, Washington, DC (US); UNIVERSITY OF SOUTH DAKOTA, Vermillion, SD (US)

(72) Inventors: Samuel Newton Sathyanesan, Vermillion, SD (US); Monica Sathyanesan, Vermillion, SD (US); Jason Petersen, Washington, DC (US)

(21) Appl. No.: 17/917,264

(22) PCT Filed: Apr. 9, 2021
(86) PCT No.: PCT/US2021/026593

§ 371 Date: Oct. 6, 2022

Related U.S. Application Data
(60) Provisional application No. 63/007,716, filed on Apr. 9, 2020.

(51) Int. Cl. C07K 14/505; A61P 25/28

(52) U.S. Cl. CPC C07K 14/505; A61P 25/28; A61K 38/00

(57) ABSTRACT

Disclosed are polypeptides comprising an engineered recombinant EPO. For example, disclosed are polypeptides comprising the sequence of SEQ ID NO: 1. Disclosed are variant Epo polypeptides comprising three amino acid substitutions at positions 20, 45 and 97 of wild type human Epo. Disclosed are polynucleotides comprising a nucleic acid capable of encoding one or more of the disclosed polypeptides. Disclosed are vectors comprising any of the polynucleotides disclosed herein. Disclosed are compositions comprising the disclosed polypeptides, polynucleotides or vectors. Disclosed are cells comprising one or more of the disclosed polypeptides, one or more of the disclosed polynucleotides, and/or one or more of the disclosed vectors. Disclosed are methods of using a therapeutically effective amount of one or more of the disclosed polypeptides, nucleic acids or vectors to a subject in need thereof.

### CROSS-REFERENCE TO RELATED APPLICATIONS

This application claims the benefit of U.S. Provisional Patent Application No. 63/007,716, filed on Apr. 9, 2020, which is incorporated by reference herein in its entirety.

### STATEMENT REGARDING FEDERALLY SPONSORED RESEARCH

This invention was made with government support under R01 MH106640 awarded by the National Institutes of Health. The government has certain rights in the invention.

### REFERENCE TO SEQUENCE LISTING

The Sequence Listing submitted Apr. 9, 2021 as a text file named “37759_0262P1_Sequence_Listing.txt,” created on Apr. 9, 2021, and having a size of 24,629 bytes is hereby incorporated by reference pursuant to 37 C.F.R. § 1.52(e)(5).

### BACKGROUND

Carbamoylated erythropoietin (CEPO) is a chemically engineered, nonhematopoietic derivative of erythropoietin that retains its antidepressant and pro-cognitive effects, which are attributed to the increased expression of neurotrophic factors, like Brain Derived Neurotrophic Factor (BDNF), in the central nervous system. However, the chemical reaction, which produces CEPO from erythropoietin (EPO), requires pure EPO as raw material and can also cause batch-to-batch variability. To remove this disadvantage while retaining its behavioral effects, the disclosed invention describes the expression and characterization of a triple-substitution polypeptide mimetic of CEPO, named QPO.

### BRIEF SUMMARY

Disclosed are polypeptides comprising an engineered recombinant EPO. For example, disclosed are polypeptides comprising the sequence

(SEQ ID NO: 1)
APPRLICDSRVLERYLLEAQEAENITTGCAEHCSLNENITVPDTQVNFY
AWKRMEVGQQAVEVWQGLALLSEAVLRGQALLVNSSQPWEPLQLHVDQA
VSGLRSLTTLLRALGAQKEAISPPDAASAAPLRTITADTFRKLFRVYSN
FLRGKLKLYTGEACRTGDR

Disclosed are variant Epo polypeptides comprising three amino acid substitutions at positions 20, 45 and 97 of wild type human Epo. In some aspects, the substitution is a lysine (K) to glutamine (Q) substitution at one or more of positions 20, 45, and 97.

Disclosed are polynucleotides comprising a nucleic acid capable of encoding one or more of the disclosed polypeptides. For example, disclosed are polynucleotides comprising the nucleic acid sequence of

(SEQ ID NO. 4)
GCTCCGCCGCGCCTGATCTGTGACTCTCGTGTCCTGGAACGCTATCTGCTG
GAAGCGCAGGAAGCCGAAAACATTACCACGGGCTGCGCCGAACATTGTAGC
CTGAACGAAAATATCACCGTTCCGGATACGCAGGTCAATTTTTATGCATGG
AAACGTATGGAAGTCGGCCAGCAAGCTGTGGAAGTTTGGCAAGGTCTGGCA
CTGCTGTCTGAAGCAGTGCTGCGTGGTCAGGCACTGCTGGTTAACAGCTCT
CAACCGTGGGAACCGCTGCAGCTGCACGTCGACCAAGCCGTGAGTGGTCTG
CGTTCCCTGACCACGCTGCTGCGTGCACTGGGTGCTCAGAAAGAAGCGATT
TCACCGCCGGATGCAGCATCGGCAGCTCCGCTGCGTACCATCACGGCAGAC
ACCTTTCGTAAACTGTTCCGCGTTTACTCCAATTTCCTGCGCGGTAAACTG
AAACTGTATACGGGTGAAGCCTGTCGCACGGGTGACCGC.

Disclosed are vectors comprising any of the polynucleotides disclosed herein.

Disclosed are compositions comprising the disclosed polypeptides, polynucleotides or vectors.

Disclosed are cells comprising one or more of the disclosed polypeptides, one or more of the disclosed polynucleotides, and/or one or more of the disclosed vectors.

Disclosed are methods of treating depression comprising administering a therapeutically effective amount one or more of the disclosed polypeptides, nucleic acids, or vectors to a subject in need thereof.

Disclosed are methods of increasing expression of neurotrophic genes comprising administering a therapeutically effective amount of one or more of the disclosed polypeptides, polynucleotides, vectors or compositions to a subject in need thereof.

Disclosed are methods of activing an EPOR comprising administering a therapeutically effective amount of one or more of the disclosed polypeptides, polynucleotides, vectors or compositions to a subject in need thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1D show design of QPO from EPO-EPOR complex. (A) EPO is shown bound to EPOR (molecular surface representation)—PDB ID-1EER. The high affinity active site 1 (AS1) is on EPOR chain B (EPOR B) and the low affinity active site 2 (AS2) is on EPOR chain C (EPOR C). (B) The front view in A is rotated 90° toward the viewer. (C) Magnified view of boxed region in A is shown. The 3 amino acid residues that were chosen for substitution mutagenesis are indicated with the corresponding residue number in the sequence. The distance between the residue atoms and the nearest receptor atom in the active sites are indicated. (D) Magnified view of the boxed region in the top view is shown.

FIGS. 2A, 2B, and 2C show a purification Silver Stain and Western Blot Analyses. (A) A silver stain depicting each step of the purification procedure is presented. From left to right with the well # in parentheses: (1) broadband protein ladder (2) the insoluble fraction of the whole cell lysate (3) the soluble fraction of the whole cell lysate (4) the eluate of the amylose binding column, containing a mixture of MBP-QPO and MBP (5) cleavage of MBP-QPO using factor Xa, resulting in the disappearance of the MBP-QPO band, and appearance of the QPO band at approximately 22 kDa (6) eluate from immobilized metal affinity chromatography column, containing the histidine-tagged QPO polypeptide with imidazole (7) dialysis of QPO polypeptide into 1× phosphate buffered saline (B) A western blot in which QPO's reactivity with an anti-EPO antibody was compared with ngEPO, dgCEPO, EPO, and CEPO is shown. (C) A western blot in which QPO's reactivity with an anti-6× histidine antibody was compared with ngEPO, dgCEPO, EPO, and CEPO is shown.

FIGS. 3A and 3B show BDNF gene expression levels in-vitro and in-vivo. (A) The fold change in expression levels of BDNF mRNA in neuronally differentiated PC-12 cells are shown in comparison to vehicle treated controls. BDNF shows a significant upregulation in expression after treatment with QPO (p<0.001). (B) The fold change in the expression of BDNF in the hippocampus of BALB/c mice 48 hrs after a 10-dose treatment regimen (see Methods and Materials) is shown. BDNF shows a statistically significant 50% increase in expression compared to vehicle treated controls (p<0.05, N=6).

FIGS. 4A, 4B, 4C, 4D, 4E, 4F, 4G, and 4H show behavioral assays and hematocrit. (A) The treatment and testing schedule for the FST and OFT is shown. (B) The cumulative immobile duration over the scored portion of the FST is shown for both QPO-treated and vehicle-treated mice. QPO treatment resulted in a significantly decreased immobile duration compared to vehicle treated controls (p<0.05, N=6). (C) The cumulative distance moved in the OFT is shown for QPO-treated and vehicle-treated mice. QPO showed no significant difference in total distance moved during the OFT compared to vehicle treated mice (p>0.05, N=6). (D) The treatment and testing schedule for the ORMT is shown. (E) The preference of QPO-treated vs vehicle-treated mice for a novel object instead of a trained object is shown here as a discrimination index. QPO showed a significantly increased preference for the novel object compared to vehicle treated controls (p<0.05, N=6 for treated, 8 for vehicle control). (F) The comparison of total hematocrit (as a percentage) between QPO-treated mice (10 doses, 40 μg/kg i.p., over 14 days) and vehicle-treated mice is shown. QPO treatment caused neither a significant increase nor decrease in hematocrit. (G) Treatment and testing schedule for mice undergoing Novelty Induced Hypophagia Testing. Idv.Cages=day that mice were separated into individual housing. HC=Home Cage, NC=Novel Cage. (H) NIHT results for QPO-treated mice are presented in comparison to vehicle treated controls in terms of the duration of time between introduction of sweetened condensed milk and the first drink in seconds. Statistical significance is denoted by *: p<0.05.

FIGS. 5A, 5B, and 5C show Binding Affinity Assessment and Dissociation Constant Calculation. This figure presents the average calculated free energies of binding (AGB) of ngEPO and QPO to (A) Active Site 1 of EPOR/EPOR and (B) Active Site 2 of EPOR/EPOR. Statistical significance is denoted by *: p<0.05 by Student's T-test. (C) This table provides calculated dissociation constants (nM) for ngEPO and QPO. Experimentally determined EPO dissociation constants taken from Goldwasser/Wilson and Jolliffe.

FIGS. 6A and 6B show recombinant EPO constructs. (A) schematic diagram of recombinant EPO. (B) plasmid construct.

FIG. 7 shows a silver stained gel of purified recombinant EPO constructs.

FIG. 8 shows PC-12 cell gene profiles.

FIG. 9 is an example of an object recognition memory test (ORMT).

FIG. 10 is an example of the forced swim test (FST) and open field test (OFT).

FIG. 11 shows the set up for a novelty induced hyophagia test (NIHT) and OFT.

FIG. 12 shows the results of the NIHT study.

FIG. 13 shows the results of an ORMT.

FIG. 14 shows the results of a FST.

FIG. 15 shows the results of an OFT.

FIG. 16 provides the non-hematopoietic effect of different treatments.

FIG. 17 shows the hippocampal gene profiles of different treatments.

FIG. 18 shows that recombinant EPO, also known as QPO, has similar antidepressant effects to CEPO.

FIG. 19 shows a schematic of the design of EnRec1.

FIGS. 20A-F show mass spectrometry (MS) mapping of CEPO. (a) CEPO was subjected to iterative MS peptide mapping (>200 peptides) using tandem LC-MS/MS to identify all carbamylated residues. Trypsin and chymotrypsin were used for digestion. Full coverage AA sequence is provided and modified residues with position number indicated are the underlined lysines (K). (b) Cb-residues are indicated in the crystal structure of EPO (ribbon structure) bound to EPOR. (c-e) Close views (front, rear and top) of CEPO showing Cb residues away from the receptor dimers and Cb-residues (with positions numbered) closest to the receptor.

FIGS. 21A-D shows active site geometry. The atomic distance was measured between the 3 key Cb-residues, K45, K20 and K97 and known, interacting receptor active site amino acids. (a) The spatial distribution of the 2 EPOR active sites are indicated in the molecular surface representation of the 2 dimer chains. The location of critical, negatively charged, acidic, receptor residues (Glu34, 62 and 202) are highlighted in green. (b-d) The distance between Cb residues and receptor active site residues are indicated, K45-E62=3.12 Å; K20−E202=3.59 Å and K97-E34=2.73 Å.

FIGS. 22A-F shows behavior of carbamylated AA. Molecular Dynamics simulations were performed in Yasara Structure to examine Cb-induced changes in receptor interactions. Simulation was at 298 K and cell filled with water (0.997 g/ml). (a-c) Superimposed structures (by overlaying alpha carbons) of EPO and CEPO are shown in relation to receptor active site residues (E202, E62 and E34). EPO carbons are in white and CEPO carbons are in orange. Hydrogens are not shown for sake of clarity. (d-f) For greater clarity ball-stick rendering in Schrodinger software is shown. EPO carbons are in sliver gray and CEPO in green. The change in atomic distance is also indicated for each pair

FIGS. 23A-C shows glutamine substitution of Cb-lysine. (a) A MD simulation cell is shown highlighting loss of interaction between Cb-K97 and E34. For sake of clarity only few waters (red-white boomerangs) are shown. (b) Close up of the interaction shows Cb-K97 moving away from E34. (c) Computational mutagenesis substituting K97 with Glutamine (Q97) reproduces the behavior of Cb-K97

FIGS. 24A-C shows EnRec1 expression construct (a) The full length human Epo gene with multiple targeted substitutions (to produce Enrecl) was cloned into the pMAL-c4x vector, downstream the malE gene of E. coli. (b) malE encodes the maltose binding protein (MBP) which strongly binds amylose and also serves to solubilize the fused protein. His and tobacco etch virus (TEV) sequences were introduced to facilitate detection and additional purification. (c) Full amino acid sequence of MBP (black), His (light grey string of six H) and TEV (underlined) and Enrecl (light grey) are shown as in the fusion construct.

FIGS. 25A-C show Enrecl expression and purification. (a) Lane 1-marker, 2-pre-IPTG induction, 3-16 h, 4-3 h at 30 C, 5-4 h at 37 C. Best yield with 4 h at 37 C (red arrow). (b) Amylose binding purification of MBP-Enrecl fusion protein. Lane 1-lysate, 2-supernatant, 3-flow thru, 4-wash, 5-wash, 6-eluate (MBP-Enrecl), 7-post elution resin, 8-marker. (c) Cleaving Enrecl from MBP. Lane 1-pure BSA, 2-marker, 3-Enrecl.

FIGS. 26A and 26B show EnRec1-induced gene regulation. Neuronal morphology PC12 cells were treated with 100 ng/ml EnRec1 for 3 h (a) or 5 h (b) and processed for QPCR analysis using gene-specific primers and Sybr green chemistry. Specificity of product was validated by melt curve analysis. Regulation was normalized using 3 housekeeping genes. Error bars=SEM, N=4 (*p<0.01).

FIGS. 27A-C show EnRec1 induces neurogenic transcription factor Ascl1. C57B16 mice were administered 20 μg/kg EnRec1 for 5 days. Hippocampal cryosections were collected on LMD slides and the DG and SGZ (1 cell layer) microdissected. (a) The dentate gyrus (DG) and SGZ (white arrows) are shown prior to SGZ dissection. A single cell layer is outlined (green line) using free draw tool in the Leica LMD7000. (b) Precise excision of outlined SGZ region (white arrows). (c) RNA was isolated and processed for determining Ascl1 gene expression using QPCR. Gene regulation from Vehicle and EnRec1 groups were normalized to housekeeping genes. Error bars=SEM, N=4 (*p<0.03).

FIGS. 28A-C show ribbon structures of the complex of EPOR C and EPOR B with EPO. The computational structural biology modeling studies shown here indicate that the sugar moieties are not involved in receptor binding. The location of glycosylation sites is indicated by cylinders along with the positions of the amino acid residues involved. EPO is depicted in dark helix configuration and EPOR is shown in light gray in the molecular surface configuration. A (front view) and B (side view) show that 3 glycosylation sites, N38, N83 and S126 are away from the receptor. C (rear view) shows that the 4th site, N24 is also away from the receptor.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a polypeptide” includes a plurality of such polypeptides, reference to “the polynucleotide” is a reference to one or more polynucleotides and equivalents thereof known to those skilled in the art, and so forth.

As used herein an amino acid “substitution” refers to the replacement of one amino acid residue by a different amino acid residue. The substituted amino acid may be any of the 20 amino acids commonly found in human proteins, as well as atypical or non-naturally occurring amino acids. A substitution of an amino acid residue can be considered conservative or non-conservative. Conservative substitutions are those within the following groups: Ser, Thr, and Cys; Leu, ILe, and Val; Glu and Asp; Lys and Arg; Phe, Tyr, and Trp; and Gln, Asn, Glu, Asp, and His. In some aspects, the substitution can be a non-naturally occurring substitution. For example, the substitution may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH2(CH₂)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “wild-type” refers to a gene or gene product which has the characteristics of that gene or gene product when isolated from a naturally-occurring source.

The terms “variant” and “mutant” are used interchangeably herein. As used herein, the term “variant” refers to a modified nucleic acid or protein which displays the same characteristics when compared to a reference nucleic acid or protein sequence. A variant can be at least 65, 70, 75, 80, 85, 90, 95, or 99 percent homologous to a reference sequence. In some aspects, a reference sequence can be a wild type EPO nucleic acid sequence or a wild type EPO protein sequence. Variants can also include nucleotide sequences that are substantially similar to sequences of Enrecl disclosed herein. A “variant” or “variant thereof” can mean a difference in some way from the reference sequence other than just a simple deletion of an N- and/or C-terminal amino acid residue or residues. Where the variant includes a substitution of an amino acid residue, the substitution can be considered conservative or non-conservative. Variants can include at least one substitution and/or at least one addition, there may also be at least one deletion. Variants can also include one or more non-naturally occurring residues.

The term “percent (%) homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res. 25:3389-3402, 1997.) A preferred alignment of selected sequences in order to determine“% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3, 4 nucleotide base changes as compared to the parent or reference sequence of the invention, and shares or improves biological function, specificity and/or activity of the parent sequence. Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence, an EPO nucleic acid sequence or EPO protein sequence).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

By an “effective amount” of a composition as provided herein is meant a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation. The term “therapeutically effective amount” means an amount of a therapeutic, prophylactic, and/or diagnostic agent (e.g., engineered recombinant EPO) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition. The term is also intended to refer to an amount of nanocarrier or composition thereof provided herein that modulates an immune response in a subject

By “treat” is meant to administer a peptide, nucleic acid, vector, or composition of the invention to a subject, such as a human or other mammal (for example, an animal model), that has an increased susceptibility for developing depression or any cognitive impairment, or that has depression or a cognitive impairment, in order to prevent or delay a worsening of the effects of the disease or condition, or to partially or fully reverse the effects of the disease or condition.

By “prevent” is meant to minimize the chance that a subject who has an increased susceptibility for developing depression or a cognitive impairment.

The term “cognitive impairment” as used herein refers to deficits in the ability to think, remember and recognize. It also can include learning and memory deficits.

As used herein, the terms “administering” and “administration” refer to any method of providing a disclosed polypeptide, polynucleotide, vector, composition, or a pharmaceutical preparation to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed conjugate so as to treat a subject or induce apoptosis. In an aspect, the skilled person can also alter or modify an aspect of an administering step so as to improve efficacy of a disclosed polypeptide, polynucleotide, vector, composition, or a pharmaceutical preparation.

As used herein, a detectable label or detectable moiety or diagnostic moiety (also imaging label, imaging agent, or imaging moiety) refers to an atom, molecule or composition, wherein the presence of the atom, molecule or composition can be directly or indirectly measured.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Polypeptides

Disclosed are polypeptides comprising an engineered recombinant EPO. In some aspects, a disclosed engineered recombinant EPO is also referred to as EnRec1. In some aspects, the EnRec1 can be referred to as QPO since it can have three amino acid substitutions where each of the three amino acids is changed to a glutamine (Q) (e.g. at positions 20, 45 and 97 of SEQ ID NO: 3). In some aspects, the EnRec1 can be referred to as RPO having K to R substitutions at positions 20, 45 and 97 of SEQ ID NO:3. The disclosed engineered recombinant EPOs are non-erythropoietic.

Disclosed are polypeptides comprising the sequence APPRLICDSRVLERYLLEAQEAENITTGCAEHCSLNENITVPDTQVNFYAWKRMEVGQQ AVEVWQGLALLSEAVLRGQALLVNSSQPWEPLQLHVDQAVSGLRSLTTLLRALGAQK EAISPPDAASAAPLRTITADTFRKLFRVYSNFLRGKLKLYTGEACRTGDR (SEQ ID NO: 1) or variants thereof.

Disclosed are polypeptides comprising a sequence having 75, 80, 85, 90, 95, or 99% identity to SEQ ID NO:1. In some aspects, a sequence having 75, 80, 85, 90, 95, or 99% identity to SEQ ID NO:1 is not different at amino acids 20, 45 and 97.

In some aspects, full length wild type human Epo is represented by the amino acid sequence of

(SEQ ID NO: 8)
MGVHECPAWLWLLLSLLSLPLGLPVLGAPPRLICDSRVLERYLLEAKEAEN
ITTGCAEHCSLNENITVPDTKVNFYAWKRMEVGQQAVEVWQGLALLSEAVL
RGQALLVNSSQPWEPLQLHVDKAVSGLSLTTLLRALGAQKEAISPPDAASA
APLRTITADTFRKLFRVYSNFLRGKLKLYTGEACRTGDR

The underlined portion represents a signal sequence. In some aspects, the disclosed polypeptides can comprise a sequence that starts at amino acid 28 of SEQ ID NO:8. In some aspects, a portion of wild type Epo can comprise the amino acid sequence of APPRLICDSRVLERYLLEAKEAENITTGCAEHCSLNENITVPDTKVNFYAWKRMEVGQQ AVEVWQGLALLSEAV LRGQALLVNSSQPWEPLQLHVDKAVSGLSLTTLLRALGA QKEAISPPDAASAAPLRTITADTFRKLFRVYSNFLRGKLK LYTGEACRTG DR (SEQ ID NO:3) or variants thereof.

Disclosed are variant Epo polypeptides comprising three amino acid substitutions at positions 20, 45 and 97 (shown in bold above) of the wild type human Epo represented as SEQ ID NO: 3.

In some aspects, the substitution is a lysine (K) to glutamine (Q) substitution at one or more of positions 20, 45, and 97. In some aspects, any polar, uncharged side chain amino acid can be substituted. In some aspects, any amino acid that simulates the charge, size and shape of the carbamoylated lysines from CEPO can be used. In some aspects, the substitution is an lysine (K) to arginine (R) substitution at one or more of positions 20, 45, and 97.

Disclosed are polypeptides comprising a sequence having 75, 80, 85, 90, 95, or 99% identity to SEQ ID NO:3, wherein at least positions 20, 45 and 97 are substituted from SEQ ID NO:3. Thus, disclosed are variants of the polypeptide of SEQ ID NO:3.

In some aspects, additional sequences can be added to the disclosed sequences to aid in expression or detection of the polypeptide.

For example, additional sequences that aid in solubilizing, detecting, and/or purifying the polypeptide can be added to the polypeptide. In some aspects, the disclosed polypeptides can further comprise a maltose binding protein sequence. A maltose binding protein can help with protein solubilization, protein detection, and protein purification In some aspects, the disclosed polypeptides can further comprise a histidine tag. A histidine tag can be used for protein purification and detection. Those of skill in the art would understand those known sequences available for solubilizing, detecting, and/or purifying polypeptides that can be used with the disclosed polypeptides. In some aspects, the disclosed polypeptides can further comprise a detectable label or diagnostic moiety.

In some aspects, a maltose binding protein sequence can be MKIEEGKLVIWINGDKGYNGLAEVGKKFEKDTGIKVTVEHPDKLEEKFPQVAATGDGP DIIFWAHDRFGGYAQSGLLAEITPDKAFQDKLYPFTWDAVRYNGKLIAYPIAVEALSLIY NKDLLPNPPKTWEEIPALDKELKAKGKSALMFNLQEPYFTWPLIAADGGYAFKYENGK YDIKDVGVDNAGAKAGLTFLVDLIKNKHMNADTDYSIAEAAFNKGETAMTINGPWAW SNIDTSKVNYGVTVLPTFKGQPSKPFVGVLSAGINAASPNKELAKEFLENYLLTDEGLEA VNKDKPLGAVALKSYEEELAKDPRIAATMENAQKGEIMPNIPQMSAFWYAVRTAVINA ASGRQTVDEALKDAQT (SEQ ID NO:7) or a variant thereof.

Disclosed are polypeptides comprising the sequence of SEQ ID NO:2 or variants thereof.

SEQ ID NO: 2 consists of
MKIEEGKLVIWINGDKGYNGLAEVGKKFEKDTGIKVTVEHPDKLEEKFPQV
AATGDGPDIIFWAHDRFGGYAQSGLLAEITPDKAFQDKLYPFTWDAVRYNG
KLIAYPIAVEALSLIYNKDLLPNPPKTWEEIPALDKELKAKGKSALMFNLQ
EPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQP
SKPFVGVLSAGINAASPNKELAKEFLENYLLTDEGLEAVNKDKPLGAVALK
SYEEELAKDPRIAATMENAQKGEIMPNIPQMSAFWYAVRTAVINAASGRQT
VDEALKDAQT_{NSSSNNNNNNNNNNLGIEG}RISEFHHHHHHAPPRLICDSRVLERYL
LEAQEAENITTGCAEHCSLNENITVPDTQVNFYAWKRMEVGQQAVEVWQG
LALLSEAVLRGQALLVNSSQPWEPLQLHVDQAVSGLRSLTTLLRALGAQK
EAISPPDAASAAPLRTITADTFRKLFRVYSNFLRGKLKLYTGEACRTGDR.

The bold amino acids represent the MBP sequence. The underlined amino acids is the location where factor Xa cleaves (between the RI residues). The italicized amino acids indicate the 6×His tag.

The double underlined amino acids represent EnRec1. The subscript amino acids indicate a sequence from the pMAL-c4x cloning vector that can include sites for the restriction enzymes such as SacI and AvaI.

Disclosed are polypeptides comprising a sequence having 75, 80, 85, 90, 95, or 99% identity to SEQ ID NO:2. Thus, disclosed are variants of polypeptides comprising SEQ ID NO:2.

In some aspects, the disclosed polypeptides can further comprise one or more of the disclosed polypeptides conjugated to a targeting moiety.

In some aspects, the disclosed polypeptides can further comprise a cleavage site. For example, the disclosed polypeptides can further comprise a Factor Xa or tobacco etch virus (TEV) peptide sequence. The presence of the cleavage site sequence call allow for cleavage, thus releasing any excess sequences, such as purification tags, from an EnRec1 sequence. In some aspects, the cleavage site sequence can be located immediately 5′ to the EnRec1 sequence. For example, a cleavage sequence can be located between the 6×His tag and EnRec1 in SEQ ID NO:2. For example, the presence of the TEV peptide sequence call allow for cleavage using a TEV protease, thus releasing any excess sequences, such as purification tags, from an EnRec1 sequence. In some aspects, a TEV peptide sequence can be ENLYFQ (SEQ ID NO:9).

In some aspects, the disclosed polypeptides lack the native carbohydrate moiety in comparison to native human erythropoietin or carbamylated erythropoietin.

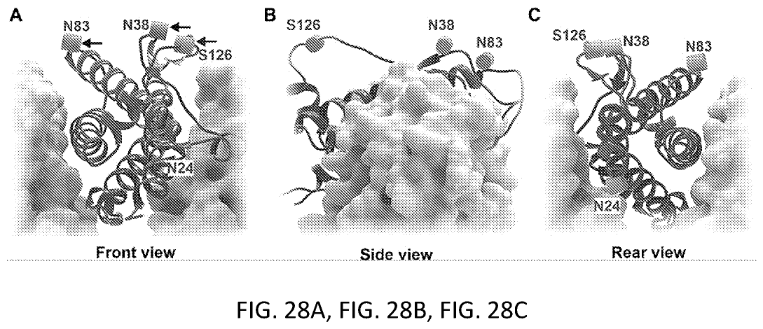

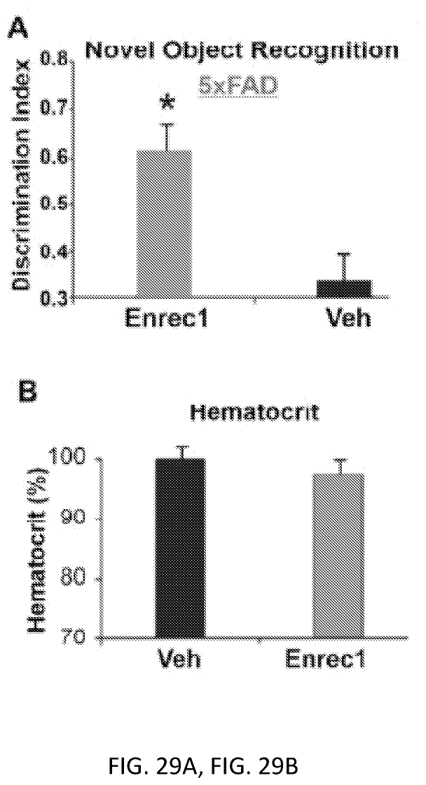

In some aspects, the disclosed polypeptides are 40% smaller in comparison to native human erythropoietin. In some aspects, the disclosed polypeptides can be 40% smaller than human EPO because they lack the sugar/carbohydrate moiety or glycosylation that comprises 40% of the molecular weight. Our computational structural biology modeling studies (see FIG. 28) indicate that the sugar moieties are not involved in receptor binding. The location of glycosylation sites is indicated by cylinders along with the positions of the amino acid residues involved. EPO is depicted in dark helix configuration and EPOR is shown in light gray in the molecular surface configuration. FIG. 28A (front view) and FIG. 28 B (side view) show that 3 glycosylation sites, N38, N83 and 5126 are away from the receptor. FIG. 28 C (rear view) shows that the 4th site, N24 is also away from the receptor. Thus, in some aspects, the disclosed polypeptides are not glycosylated.

In some aspects, the disclosed polypeptides can cross the blood brain barrier (BBB). The ability to cross the BBB allows for the disclosed polypeptides to act on the brain and produce behavioral effects. In some aspects, the disclosed polypeptides can further comprise a targeting moiety. In some aspects, the targeting moiety can direct, or target, the polypeptide across the BBB. The targeting moiety can be a chemical, compound, peptide or nucleic acid. Examples of targeting moieties include, but art not limited to, molecules that recognize receptors present in the BBB (e.g., LDLR, transferrin receptor, insulin-like growth factor receptor), antibodies,

### C. Nucleic Acids

Disclosed are polynucleotides comprising a nucleic acid capable of encoding one or more of the disclosed polypeptides.

In some aspects, disclosed are polynucleotides comprising the nucleic acid sequence of GCTCCGCCGCGCCTGATCTGTGACTCTCGTGTCCTGGAACGCTATCTGCTGGAAGCG CAGGAAGCCGAAAACATTACCACGGGCTGCGCCGAACATTGTAGCCTGAACGAAA ATATCACCGTTCCGGATACGCAGGTCAATTTTTATGCATGGAAACGTATGGAAGTCG GCCAGCAAGCTGTGGAAGTTTGGCAAGGTCTGGCACTGCTGTCTGAAGCAGTGCTG CGTGGTCAGGCACTGCTGGTTAACAGCTCTCAACCGTGGGAACCGCTGCAGCTGCA CGTCGACCAAGCCGTGAGTGGTCTGCGTTCCCTGACCACGCTGCTGCGTGCACTGGG TGCTCAGAAAGAAGCGATTTCACCGCCGGATGCAGCATCGGCAGCTCCGCTGCGTA CCATCACGGCAGACACCTTTCGTAAACTGTTCCGCGTTTACTCCAATTTCCTGCGCG GTAAACTGAAACTGTATACGGGTGAAGCCTGTCGCACGGGTGACCGC (SEQ ID NO. 4) or variants thereof. SEQ ID NO:4 represents the nucleic acid sequence that encodes for EnRec1.

In some aspects, disclosed are polynucleotides comprising the nucleic acid sequence of

(SEQ ID NO: 5)
CCGACACCATCGAATGGTGCAAAACCTTTCGCGGTATGGCATGATAGCGCC
CGGAAGAGAGTCAATTCAGGGTGGTGAATGTGAAACCAGTAACGTTATACG
ATGTCGCAGAGTATGCCGGTGTCTCTTATCAGACCGTTTCCCGCGTGGTGA
ACCAGGCCAGCCACGTTTCTGCGAAAACGCGGGAAAAAGTGGAAGCGGCGA
TGGCGGAGCTGAATTACATTCCCAACCGCGTGGCACAACAACTGGCGGGCA
AACAGTCGTTGCTGATTGGCGTTGCCACCTCCAGTCTGGCCCTGCACGCGC
CGTCGCAAATTGTCGCGGCGATTAAATCTCGCGCCGATCAACTGGGTGCCA
GCGTGGTGGTGTCGATGGTAGAACGAAGCGGCGTCGAAGCCTGTAAAGCGG
CGGTGCACAATCTTCTCGCGCAACGCGTCAGTGGGCTGATCATTAACTATC
CGCTGGATGACCAGGATGCCATTGCTGTGGAAGCTGCCTGCACTAATGTTC
CGGCGTTATTTCTTGATGTCTCTGACCAGACACCCATCAACAGTATTATTT
TCTCCCATGAAGACGGTACGCGACTGGGCGTGGAGCATCTGGTCGCATTGG
GTCACCAGCAAATCGCGCTGTTAGCGGGCCCATTAAGTTCTGTCTCGGCGC
GTCTGCGTCTGGCTGGCTGGCATAAATATCTCACTCGCAATCAAATTCAGC
CGATAGCGGAACGGGAAGGCGACTGGAGTGCCATGTCCGGTTTTCAACAAA
CCATGCAAATGCTGAATGAGGGCATCGTTCCCACTGCGATGCTGGTTGCCA
ACGATCAGATGGCGCTGGGCGCAATGCGCGCCATTACCGAGTCCGGGCTGC
GCGTTGGTGCGGATATCTCGGTAGTGGGATACGACGATACCGAAGACAGCT
CATGTTATATCCCGCCGTTAACCACCATCAAACAGGATTTTCGCCTGCTGG
GGCAAACCAGCGTGGACCGCTTGCTGCAACTCTCTCAGGGCCAGGCGGTGA
AGGGCAATCAGCTGTTGCCCGTCTCACTGGTGAAAAGAAAAACCACCCTGG
CGCCCAATACGCAAACCGCCTCTCCCCGCGCGTTGGCCGATTCATTAATGC
AGCTGGCACGACAGGTTTCCCGACTGGAAAGCGGGCAGTGAGCGCAACGCA
ATTAATGTAAGTTAGCTCACTCATTAGGCACAATTCTCATGTTTGACAGCT
TATCATCGACTGCACGGTGCACCAATGCTTCTGGCGTCAGGCAGCCATCGG
AAGCTGTGGTATGGCTGTGCAGGTCGTAAATCACTGCATAATTCGTGTCGC
TCAAGGCGCACTCCCGTTCTGGATAATGTTTTTTGCGCCGACATCATAACG
GTTCTGGCAAATATTCTGAAATGAGCTGTTGACAATTAATCATCGGCTCGT
ATAATGTGTGGAATTGTGAGCGGATAACAATTTCACACAGGAAACAGCCAG
TCCGTTTAGGTGTTTTCACGAGCACTTCACCAACAAGGACCATAGATTATG
AAAATCGAAGAAGGTAAACTGGTAATCTGGATTAACGGCGATAAAGGCTAT
AACGGTCTCGCTGAAGTCGGTAAGAAATTCGAGAAAGATACCGGAATTAAA
GTCACCGTTGAGCATCCGGATAAACTGGAAGAGAAATTCCCACAGGTTGCG
GCAACTGGCGATGGCCCTGACATTATCTTCTGGGCACACGACCGCTTTGGT
GGCTACGCTCAATCTGGCCTGTTGGCTGAAATCACCCCGGACAAAGCGTTC
CAGGACAAGCTGTATCCGTTTACCTGGGATGCCGTACGTTACAACGGCAAG
CTGATTGCTTACCCGATCGCTGTTGAAGCGTTATCGCTGATTTATAACAAA
GATCTGCTGCCGAACCCGCCAAAAACCTGGGAAGAGATCCCGGCGCTGGAT
AAAGAACTGAAAGCGAAAGGTAAGAGCGCGCTGATGTTCAACCTGCAAGAA
CCGTACTTCACCTGGCCGCTGATTGCTGCTGACGGGGGTTATGCGTTCAAG
TATGAAAACGGCAAGTACGACATTAAAGACGTGGGCGTGGATAACGCTGGC
GCGAAAGCGGGTCTGACCTTCCTGGTTGACCTGATTAAAAACAAACACATG
AATGCAGACACCGATTACTCCATCGCAGAAGCTGCCTTTAATAAAGGCGAA
ACAGCGATGACCATCAACGGCCCGTGGGCATGGTCCAACATCGACACCAGC
AAAGTGAATTATGGTGTAACGGTACTGCCGACCTTCAAGGGTCAACCATCC
AAACCGTTCGTTGGCGTGCTGAGCGCAGGTATTAACGCCGCCAGTCCGAAC
AAAGAGCTGGCAAAAGAGTTCCTCGAAAACTATCTGCTGACTGATGAAGGT
CTGGAAGCGGTTAATAAAGACAAACCGCTGGGTGCCGTAGCGCTGAAGTCT
TACGAGGAAGAGTTGGCGAAAGATCCACGTATTGCCGCCACCATGGAAAAC
GCCCAGAAAGGTGAAATCATGCCGAACATCCCGCAGATGTCCGCTTTCTGG
TATGCCGTGCGTACTGCGGTGATCAACGCCGCCAGCGGTCGTCAGACTGTC
GATGAAGCCCTGAAAGACGCGCAGACTAATTCGAGCTCGAACAACAACAAC
AATAACAATAACAACAACCTCGGGATCGAGGGAAGGATTTCAGAATTCCAT
CACCATCATCACCACGCTCCGCCGCGCCTGATCTGTGACTCTCGTGTCCTG
GAACGCTATCTGCTGGAAGCGCAGGAAGCCGAAAACATTACCACGGGCTGC
GCCGAACATTGTAGCCTGAACGAAAATATCACCGTTCCGGATACGCAGGTC
AATTTTTATGCATGGAAACGTATGGAAGTCGGCCAGCAAGCTGTGGAAGTT
TGGCAAGGTCTGGCACTGCTGTCTGAAGCAGTGCTGCGTGGTCAGGCACTG
CTGGTTAACAGCTCTCAACCGTGGGAACCGCTGCAGCTGCACGTCGACCAA
GCCGTGAGTGGTCTGCGTTCCCTGACCACGCTGCTGCGTGCACTGGGTGCT
CAGAAAGAAGCGATTTCACCGCCGGATGCAGCATCGGCAGCTCCGCTGCGT
ACCATCACGGCAGACACCTTTCGTAAACTGTTCCGCGTTTACTCCAATTTC
CTGCGCGGTAAACTGAAACTGTATACGGGTGAAGCCTGTCGCACGGGTGAC
CGCTGAGGATCCTCTAGAGTCGACCTGCAGGCAAGCTTGGCACTGGCCGTC
GTTTTACAACGTCGTGACTGGGAAAACCCTGGCGTTACCCAACTTAATCGC
CTTGCAGCACATCCCCCTTTCGCCAGCTGGCGTAATAGCGAAGAGGCCCGC
ACCGATCGCCCTTCCCAACAGTTGCGCAGCCTGAATGGCGAATGGCAGCTT
GGCTGTTTTGGCGGATGAGATAAGATTTTCAGCCTGATACAGATTAAATCA
GAACGCAGAAGCGGTCTGATAAAACAGAATTTGCCTGGCGGCAGTAGCGCG
GTGGTCCCACCTGACCCCATGCCGAACTCAGAAGTGAAACGCCGTAGCGCC
GATGGTAGTGTGGGGTCTCCCCATGCGAGAGTAGGGAACTGCCAGGCATCA
AATAAAACGAAAGGCTCAGTCGAAAGACTGGGCCTTTCGTTTTATCTGTTG
TTTGTCGGTGAACGCTCTCCTGAGTAGGACAAATCCGCCGGGAGCGGATTT
GAACGTTGCGAAGCAACGGCCCGGAGGGTGGCGGGCAGGACGCCCGCCATA
AACTGCCAGGCATCAAATTAAGCAGAAGGCCATCCTGACGGATGGCCTTTT
TGCGTTTCTACAAACTCTTTTGTTTATTTTTCTAAATACATTCAAATATGT
ATCCGCTCATGAGACAATAACCCTGATAAATGCTTCAATAATATTGAAAAA
GGAAGAGTATGAGTATTCAACATTTCCGTGTCGCCCTTATTCCCTTTTTTG
CGGCATTTTGCCTTCCTGTTTTTGCTCACCCAGAAACGCTGGTGAAAGTAA
AAGATGCTGAAGATCAGTTGGGTGCACGAGTGGGTTACATCGAACTGGATC
TCAACAGCGGTAAGATCCTTGAGAGTTTTCGCCCCGAAGAACGTTCTCCAA
TGATGAGCACTTTTAAAGTTCTGCTATGTGGCGCGGTATTATCCCGTGTTG
ACGCCGGGCAAGAGCAACTCGGTCGCCGCATACACTATTCTCAGAATGACT
TGGTTGAGTACTCACCAGTCACAGAAAAGCATCTTACGGATGGCATGACAG
TAAGAGAATTATGCAGTGCTGCCATAACCATGAGTGATAACACTGCGGCCA
ACTTACTTCTGACAACGATCGGAGGACCGAAGGAGCTAACCGCTTTTTTGC
ACAACATGGGGGATCATGTAACTCGCCTTGATCGTTGGGAACCGGAGCTGA
ATGAAGCCATACCAAACGACGAGCGTGACACCACGATGCCTGTAGCAATGG
CAACAACGTTGCGCAAACTATTAACTGGCGAACTACTTACTCTAGCTTCCC
GGCAACAATTAATAGACTGGATGGAGGCGGATAAAGTTGCAGGACCACTTC
TGCGCTCGGCCCTTCCGGCTGGCTGGTTTATTGCTGATAAATCTGGAGCCG
GTGAGCGTGGGTCTCGCGGTATCATTGCAGCACTGGGGCCAGATGGTAAGC
CCTCCCGTATCGTAGTTATCTACACGACGGGGAGTCAGGCAACTATGGATG
AACGAAATAGACAGATCGCTGAGATAGGTGCCTCACTGATTAAGCATTGGT
AACTGTCAGACCAAGTTTACTCATATATACTTTAGATTGATTTACCCCGGT
TGATAATCAGAAAAGCCCCAAAAACAGGAAGATTGTATAAGCAAATATTTA
AATTGTAAACGTTAATATTTTGTTAAAATTCGCGTTAAATTTTTGTTAAAT
CAGCTCATTTTTTAACCAATAGGCCGAAATCGGCAAAATCCCTTATAAATC
AAAAGAATAGCCCGAGATAGGGTTGAGTGTTGTTCCAGTTTGGAACAAGAG
TCCACTATTAAAGAACGTGGACTCCAACGTCAAAGGGCGAAAAACCGTCTA
TCAGGGCGATGGCCCACTACGTGAACCATCACCCAAATCAAGTTTTTTGGG
GTCGAGGTGCCGTAAAGCACTAAATCGGAACCCTAAAGGGAGCCCCCGATT
TAGAGCTTGACGGGGAAAGCCGGCGAACGTGGCGAGAAAGGAAGGGAAGAA
AGCGAAAGGAGCGGGCGCTAGGGCGCTGGCAAGTGTAGCGGTCACGCTGCG
CGTAACCACCACACCCGCCGCGCTTAATGCGCCGCTACAGGGCGCGTAAAA
GGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAAA
AACCACCGCTACCAGCGGTGGTTTGTTTGCCGGATCAAGAGCTACCAACTC
TTTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCGC
CTACATACCTCGCTCTGCTAATCCTGTTACCAGTGGCTGCTGCCAGTGGCG
ATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGC
GAACGACCTACACCGAACTGAGATACCTACAGCGTGAGCTATGAGAAAGCG
CCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCT
TTTTACGGTTCCTGGCCTTTTGCTGGCCTTTTGCTCACATGTTCTTTCCTG
CGTTATCCCCTGATTCTGTGGATAACCGTATTACCGCCTTTGAGTGAGCTG
ATACCGCTCGCCGCAGCCGAACGACCGAGCGCAGCGAGTCAGTGAGCGAGG
AAGCGGAAGAGCGCCTGATGCGGTATTTTCTCCTTACGCATCTGTGCGGTA
TTTCACACCGCATATGGTGCACTCTCAGTACAATCTGCTCTGATGCCGCAT
AGTTAAGCCAGTATACACTCCGCTATCGCTACGTGACTGGGTCATGGCTGC
GCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTG
TCAGAGGTTTTCACCGTCATCACCGAAACGCGCGAGGCAGCTGCGGTAAAG
CTCATCAGCGTGGTCGTGCAGCGATTCACAGATGTCTGCCTGTTCATCCGC
GTCCAGCTCGTTGAGTTTCTCCAGAAGCGTTAATGTCTGGCTTCTGATAAA
GCGGGCCATGTTAAGGGCGGTTTTTTCCTGTTTGGTCACTGATGCCTCCGT
GTAAGGGGGATTTCTGTTCATGGGGGTAATGATACCGATGAAACGAGAGAG
GATGCTCACGATACGGGTTACTGATGATGAACATGCCCGGTTACTGGAACG
TTGTGAGGGTAAACAACTGGCGGTATGGATGCGGCGGGACCAGAGAAAAAT
CACTCAGGGTCAATGCCAGCGCTTCGTTAATACAGATGTAGGTGTTCCACA
GGGTAGCCAGCAGCATCCTGCGATGCAGATCCGGAACATAATGGTGCAGGG
CGCTGACTTCCGCGTTTCCAGACTTTACGAAACACGGAAACCGAAGACCAT
TCATGTTGTTGCTCAGGTCGCAGACGTTTTGCAGCAGCAGTCGCTTCACGT
TCGCTCGCGTATCGGTGATTCATTCTGCTAACCAGTAAGGCAACCCCGCCA
GCCTAGCCGGGTCCTCAACGACAGGAGCACGATCATGCGCACCCGTGGCCA
GGACCCAACGCTGCCCGAAATT

or variant thereof. SEQ ID NO:5 represents an expression construct with EnRec1 downstream of the malE genes that encodes a maltose binding protein (MBP). In SEQ ID NO: 5, the vector is underlined; MBP sequence is underlined and bolded; His tag is in italics; ENREC is shaded and the substitutions are bolded.

In some aspects, disclosed are polynucleotides comprising the nucleic acid sequence of atggggg tgcacgaatg tcctgcctgg ctgtggcttc tcctgtccct gctgtcgctc cctctgggcc tcccagtcct gggcgcccca ccacgcctca tctgtgacag ccgagtcctg gagaggtacc tcttggaggc caaggaggcc gagaatatca cgacgggctg tgctgaacac tgcagcttga atgagaatat cactgtccca gacaccaaag ttaatttcta tgcctggaag aggatggagg tcgggcagca ggccgtagaa gtctggcagg gcctggccct gctgtcggaa gctgtcctgc ggggccaggc cctgttggtc aactcttccc agccgtggga gcccctgcag ctgcatgtgg ataaagccgt cagtggcctt cgcagcctca ccactctgct tcgggctctg ggagcccaga aggaagccat ctcccctcca gatgcggcct cagctgctcc actccgaaca atcactgctg acactttccg caaactcttc cgagtctact ccaatttcct ccggggaaag ctgaagctgt acacagggga ggcctgcagg acaggggaca gatga (SEQ ID NO:6) or variant thereof. SEQ ID NO:6 represents the nucleic acid sequence that encodes for wild type human EPO. SEQ ID NO:6 is NCBI reference sequence NM 000799.4.

In some aspects, additional sequences can be added to the disclosed sequences to aid in expression of the polypeptide. For example, additional sequences that aid in solubilizing, detecting, and/or purifying the polypeptide can be added. In some aspects, the disclosed polynucleotides can further comprise a nucleic acid sequence that encodes a maltose binding protein. In some aspects, the disclosed polynucleotides can further comprise a nucleic acid sequence that encodes for a histidine tag.

In some aspects, the disclosed polynucleotides can further comprise a cleavage site. For example, the disclosed polynucleotides can further comprise a nucleic acid sequence that encodes for a tobacco etch virus (TEV) peptide sequence, wherein the TEV peptide sequence is a cleavage site.

### D. Vectors

Disclosed are vectors comprising any of the polynucleotides disclosed herein.

The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be an adeno-associated viral vector. In some aspects, the vector can be a non-viral vector, such as a DNA based vector.

1. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding encoding a VMD2 promoter operably linked to a nucleic acid sequence encoding Rap1a.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, a-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes B-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Feigner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### E. Compositions

Disclosed are compositions comprising the disclosed polypeptides, polynucleotides or vectors. Disclosed are compositions comprising an engineered recombinant EPO. Disclosed are compositions comprising a nucleic acid construct, wherein the nucleic acid construct comprises a nucleic acid sequence encoding an engineered recombinant EPO. Also disclosed are compositions comprising a vector, such as a viral vector, comprising a nucleic acid construct, wherein the nucleic acid construct comprises a nucleic acid sequence encoding an engineered recombinant EPO.

The disclosed compositions can further comprise a pharmaceutically acceptable carrier.

1. Delivery of Compositions

In the methods described herein, delivery (or administration) of the compositions to cells can be via a variety of mechanisms. As defined above, disclosed herein are compositions comprising any one or more of the peptides, nucleic acids, and/or vectors described herein can be used to produce a composition which can also include a carrier such as a pharmaceutically acceptable carrier. For example, disclosed are pharmaceutical compositions, comprising the peptides disclosed herein, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed nucleic acid constructs and vectors.

### F. Recombinant Cells

Disclosed are cells comprising one or more of the disclosed polypeptides, one or more of the disclosed polynucleotides, and/or one or more of the disclosed vectors.

In some aspects, the cell is a bacterial cell. In some aspects, the cell can be a eukaryotic cell, such as an insect cell or mammalian cell.

### G. Methods of Treating

Disclosed are methods of treating depression comprising administering a therapeutically effective amount one or more of the disclosed polypeptides, nucleic acids, or vectors to a subject in need thereof. In some aspects, disclosed are methods of treating alzheimer's disease comprising administering a therapeutically effective amount one or more of the disclosed polypeptides, nucleic acids, or vectors to a subject in need thereof. In some aspects, treating can include improving a symptom of the disease such as improving cognitive function because cognitive function can decline in both depression and alzheimer's disease.

Disclosed are methods of treating a cognitive impairment comprising administering a therapeutically effective amount one or more of the disclosed polypeptides, nucleic acids, or vectors to a subject in need thereof. In some aspects, a cognitive impairment includes, but is not limited to, learning and memory impairments, attention impairments, spatial memory impairments, working memory and recognition memory impairments.

Disclosed are methods of treating a psychiatric disorder comprising administering a therapeutically effective amount one or more of the disclosed polypeptides, nucleic acids, or vectors to a subject in need thereof. In some aspects, a psychiatric disorder includes, but is not limited to, depressive disorder and schizophrenia.

Disclosed are methods of improving cognitive function comprising administering a therapeutically effective amount one or more of the disclosed polypeptides, nucleic acids, or vectors to a subject in need thereof. In some aspects, improving cognitive function includes, but is not limited to, increasing levels of neurotrophic factors, such as BDNF, in the hippocampus.

In some aspects, a second therapeutic can further be administered with the one or more of the disclosed polypeptides, nucleic acids, or vectors. In some aspects, the second therapeutic can be a known anti-depressant or anti-psychotic.

In some aspects, the disclosed methods further comprise monitoring of hematological parameters. Hematological parameters can include, but are not limited to, hemoglobin, mean corpuscular volume, mean corpuscular hemoglobin concentration, red blood cell distribution width, reticulocyte number and platelet count. In some aspects, the subject's red blood cell indices can be monitored. In some aspects, the methods further comprise maintaining the subject's red blood cell indices at substantially normal levels during treatment. Thus, in some aspects the red blood cell indices can be monitored and the subject's red blood cell indices can be maintained at substantially normal levels during treatment.

### H. Methods of Increasing Expression of Neurotrophic Genes

Disclosed are methods of increasing expression of neurotrophic genes comprising administering a therapeutically effective amount of one or more of the disclosed polypeptides, polynucleotides, vectors or compositions to a subject in need thereof. For example, disclosed are methods of increasing expression of neurotrophic genes comprising administering a therapeutically effective amount of one or more of the disclosed EnRec1 polypeptides. In some aspects, neurotrophic genes can be, but are not limited to, those genes that encode Achaete-scute homolog 1 (Ascl1), brain-derived neurotrophic factor (BDNF), VGF, neuritin, insulin-like growth factor 1 (IGF1), insulin-like growth factor 2 (IGF2), Midkine, GDNF, and FGF2.

In some aspects, the method further comprises administering a combination of trophic factors. For example, the disclosed methods can comprise administering a therapeutically effective amount of one or more of the disclosed EnRec1 polypeptides and insulin-like growth factor 1 (IGF-1).

In some aspects, the disclosed methods further comprise monitoring of hematological parameters. Hematological parameters can include, but are not limited to, hemoglobin, mean corpuscular volume, mean corpuscular hemoglobin concentration, red blood cell distribution width, reticulocyte number and platelet count. In some aspects, the subject's red blood cell indices can be monitored. In some aspects, the methods further comprise maintaining the subject's red blood cell indices at substantially normal levels during treatment. Thus, in some aspects the red blood cell indices can be monitored and the subject's red blood cell indices can be maintained at substantially normal levels during treatment.

### I. Methods of Activating an EPO Receptor (EPOR)

Disclosed are methods of activing an EPOR comprising administering a therapeutically effective amount of one or more of the disclosed polypeptides, polynucleotides, vectors or compositions to a subject in need thereof.

In some aspects, the activation of EPOR is required for the disclosed polypeptides, such as EnRec, to increase BNDF.

In some aspects, the disclosed methods further comprise monitoring of hematological parameters. Hematological parameters can include, but are not limited to, hemoglobin, mean corpuscular volume, mean corpuscular hemoglobin concentration, red blood cell distribution width, reticulocyte number and platelet count. In some aspects, the subject's red blood cell indices can be monitored. In some aspects, the methods further comprise maintaining the subject's red blood cell indices at substantially normal levels during treatment. Thus, in some aspects the red blood cell indices can be monitored and the subject's red blood cell indices can be maintained at substantially normal levels during treatment.

### J. Methods of Making

Disclosed are methods of making a variant Epo polypeptide.

Disclosed are methods of making a variant Epo polypeptide, wherein the variant Epo polypeptide comprises three amino acid substitutions at positions 20, 45 and 97 of SEQ ID NO: 3, the method comprising administering one of the disclosed polynucleotides or vectors comprising the disclosed polynucleotides to a cell and culturing the cell under conditions that allow for expression of the polypeptide encoded by the polynucleotide administered to the cell.

### K. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising an engineered recombinant EPO. The kits also can contain a vector.

### Examples

### A. The In-Silico, In-Vitro, and In-Vivo Analysis of a Triple Substitution Recombinant Trophic Factor Molecule

1. Introduction

Depression is the largest cause of disability in the world. Its detrimental effects on cognition and mood cause both a severe economic burden ($1 trillion/year according to the World Health Organization) and a danger to human life via suicidal ideation. Though many pharmacological options exist for this disorder, most work by the same or similar mechanisms (modulation of norepinephrine/epinephrine/dopamine levels) which are ineffective in 30% of affected individuals. The global “years lived with disability” due to depression has been consistently increasing (rising 14.1% from 2011-2017), and it has become apparent that the field needs not just a new medication, but a new class of medications. Erythropoietin (EPO) and its derivatives have emerged as strong candidates for the development of this new “class” of neurotrophic therapeutic drugs.

EPO has robust pro-cognitive and antidepressant effects which are believed to be mediated by an increase in the expression of neurotrophic factors in the hippocampal region of the brain, particularly brain derived neurotrophic factor (BDNF). In order to facilitate the use of EPO for CNS indications it is necessary to eliminate its hematopoietic effects, which prevent it from being used long-term. In the past, both the hematopoietic and neuroactive effects were believed to be mediated by the same receptor complex: the erythropoietin receptor homodimer (EPOR/EPOR). EPO binds two EPOR monomers at distinct active sites, the high affinity Active Site 1 (AS1, KD=˜1-5 nm) and the low affinity Active Site 2 (AS2, KD=˜1 μM), to draw the extracellular domains of EPOR/EPOR into a specific angular configuration that activated its downstream signaling. However, recent studies have implied that the neuroactive effects of EPO may be mediated by a heterodimer receptor comprised of an EPOR monomer and CD131 (EPOR/CD131), forming what is called the Innate Repair Receptor (IRR).

The existence of the IRR is supported by the binding affinity measurements of EPO's carbamoylated form (CEPO), in which the seven surface lysine residues are changed to homocitrulline. CEPO loses the ability to activate the EPOR/EPOR homodimer and has no hematopoietic activity in-vivo or in-vitro. However, CEPO retains EPO's neuroactive affects, implying that not only is EPOR/EPOR not mediating these effects, but also that CEPO is a selective agonist of the IRR. While this would normally make it a strong candidate for clinical testing, its expensive nonenzymatic synthesis from EPO causes significant batch-to-batch variability, limiting the ability to scale up and produce large, pure quantities of the material. CEPO is also difficult to computationally model, due to the lack of a solved crystal structure which addresses its n-linked glycosides. To address these shortcomings, a mimetic form of CEPO was expressed in E. coli, replacing the homocitrulline groups with a chemically similar amino acid (glutamine) and removing the glycosylation groups to facilitate faster systemic removal and better computational modeling.

The mimetic, called QPO, was designed to specifically mimic CEPO's inability to activate the EPOR homodimer, while retaining both its and EPO's ability to activate the EPOR/CD131 heterodimer. Therefore, only the surface lysine residues involved in binding the homodimer were altered. This was to ensure the final structure remains as similar as possible to EPO and isolate any observed changes in behavior and gene expression to a relatively small structural change. Only three of the 7 homocitrulline groups of CEPO are near the active sites of the EPO-EPOR/EPOR complex (K20, K45, K97), and so these were the only ones substituted with glutamine in QPO.

Using a neuronally differentiated Pheochromocytoma-12 (PC-12) cell line, stress-sensitive BALB/c mice, and molecular dynamics simulations based on previously reported crystal structures of the active EPO-EPOR/EPOR complex, QPO was tested for its effects on antidepressant-like behavior, cognitive function, hematopoietic activity, BDNF regulation, and EPOR/EPOR binding modality and affinity.

2. Methods:

i. Animals

Adult, male, BALB/C mice (ENVIGO) were housed in 36×15×12 cm mouse cages with free access to food and water except when undergoing behavioral testing. Animal use procedures were in strict accordance with the guidelines set forth by the University of South Dakota Sanford School of Medicine IACUC and NIH guidelines.

ii. Cell Maintenance and Treatment

Pheochromocytoma 12 (PC-12) cells were grown in RPMI 1640 (ATCC #30-2001) medium containing 5% fetal bovine serum (Gibco #A31604-01) and 10% inactivated horse serum (Gibco #26050-070). Cells were plated on 60 mm Type IV Collagen plates (Corning #62405-644) at a density of 3*105 cells/plate in 4 mL of RPMI 1640 medium and neuronally differentiated using 100 ng/mL mouse NGF 7S (Alomone Labs #N-130) and 1% inactivated horse serum. The differentiation medium was replaced every 48 hours for 10 days to allow cells to obtain maximum neurite outgrowth. On day 10, the cells were changed into medium containing no NGF for 24 hrs. prior to treatment. Cells were treated with 100 ng/mL of QPO in RPMI 1640 medium and allowed to incubate at 37° C. for 3 hours. Cells were then scraped, lysed and processed for RNA isolation and reverse transcriptase quantitative PCR analysis (RT-qPCR).

iii. Vector Design and Expression:

A DNA fragment was synthesized to encode QPO with an N-terminal 6×-Histidine tag and 5′ EcoRI and 3′ BamHI restriction sites (Integrated DNA Technologies). The 6×-His QPO DNA fragment was cloned into the pCR2.1 cloning plasmid (Invitrogen), sequenced for accuracy (Iowa State University, Office of Biotechnology), and subsequently cloned into pMal-c2 (New England Biolabs) using EcoRI and BamHI. The resulting plasmid was transformed into T7 Express Competent E. coli (New England Biolabs). Transformants were cultured in LB+ampicillin (50 ug/ml) and induced for expression with 0.4 mM Isopropyl 0-D thiogalactopyranoside (IPTG), resulting in a Maltose Binding Protein (MBP)/6×-His QPO fusion protein.

iv. Purification and Analysis:

MBP/6×-His QPO expression cell pellets from 300 ml cultures were resuspended and lysed in 10 ml Amylose Column Buffer (20 mM Tris pH=7.4, 200 mM NaCl, 1 mM EDTA in Type 1 H2O) using an Omni Bead Ruptor Homogenizer (OMNI International) (S=6.00, T=0:30, C=03, D=0:10) with 0.1 mm glass beads. Following centrifugation (16,000×g, 15 min, 4° C.), the soluble fraction was rocked at 4° C. for 1 hour with 5 ml amylose resin (New England Biolabs) for batch binding. The resin was washed 3× with 40 ml Amylose Column Buffer (centrifugation 500×g, 5 min at 4° C.) and transferred to a Poly-Prep Chromatography Column (BioRad) for elution of MBP/6×-His QPO with 10 mM maltose in Amylose Column Buffer. The eluate was concentrated using Vivaspin 10 kDal MWCO Polyethersulfone concentrator columns (Millipore Sigma) with centrifugation (4,000×g, 4° C., 5×10 minutes).

After dilution to 1 mg/ml, the MBP/6×-His QPO protein was digested with Factor Xa Protease (New England Biolabs) (18 hours at 25° C.) to cleave MBP from 6×-His QPO. The digested sample was loaded onto a Histrap FF IMAC column (General Electric) in binding buffer (20 mMol Imidazole in PBS, pH=7.4). The manufacturer's instructions were followed as written, with flow rates adjusted to 0.5 mL/min to minimize backpressure. The protein was eluted with 0.5M Imidazole (pH=7.4) in PBS, and each fraction was assayed for presence of the protein and level of purity by Coomassie and Silver Staining. All fractions containing the eluted protein were dialyzed for 15 hrs. at 4° C. into phosphate buffered saline using a 7kDal MWCO Slide-A-Lyzer dialysis casette (Thermo Scientific #66370). Final protein concentration and yield was determined by performing a protein 280 nm absorbance assay on a Nanodrop 2000 Spectrophotometer.

v. RNA isolation, cDNA Synthesis, and RT-qPCR:

RNA isolation was carried out using an Invitrogen RNAqueous Phenol-Free Total RNA Isolation Kit (AM-1912) following the manufacturer's instructions.

For cDNA synthesis, a microtube containing 50Ong RNA (volume of 1-12 uL), 1 uL Oligo DT20 Primer (Life Tech #18418020), and RNase free water (Ambion #AM9937) for a final volume of 13 uL was heated for 10 minutes at 80° C. The solution was placed on ice for 5 minutes, after which 4 μL 5XRT buffer, 1 μL 10 mM dNTP mix (Life Tech #18427088), 1 uL SuperScript III Reverse Transcriptase (Life Tech #18080044), and 1 μL SUPERase Inhibitor (Life Tech #AM2696) were added to each sample. The solution was mixed in a microfuge before incubating for 2 hours at 42° C. The synthesis was stopped through the addition of 3.5 μL of a 0.5M NaOH/50 mM EDTA solution in nuclease free water. The resulting mix was incubated at 65° C. for 10 minutes to denature DNA/RNA hybrids before neutralizing with 54 of 1M Tris-HCL (Life Tech #15567-027). At this point, 70.5 μL 10 mM Tris/1 mM EDTA (Thermo Scientific #17890) was added to each tube before adding 3 μL of 5 mg/mL acrylamide, 4 μL 5 M NaCl, and 400 μl of 100% EtOH. The resulting mixture is incubated overnight at −20° C. to precipitate the cDNA.

The precipitated cDNA was then pelleted in an Eppendorf 5804 R centrifuge (10,000 RPM, 15 minutes, 4° C.), washed with 4504 of cold 70% EtOH, pelleted again at the same settings, and dried at 65° C. The dry cDNA was then reconstituted in 1004 nuclease free water.

RT-qPCR was performed using an Eppendorf Mastercycler Realplex 2. Primers were obtained from Integrated DNA technologies. Quantification was performed using SYBR Green chemistry (Invitrogen #11762-500).

vi. In-Vivo Hippocampal Gene Expression and Hematocrit Measurement:

After completion of the Object Recognition Memory Test, the mice were allowed 2 days of rest followed by 6 additional treatments of QPO (40 μg/kg i.p. daily) for a total of 10 doses. QPO treated mice were then sacrificed 48 hrs. after the final dose via rapid decapitation, during which truncal blood was collected for hematocrit measurement in Microvette CB 300 μL K2 EDTA tubes (Sarstedt #16.444). The hippocampus was dissected out and processed for RNA isolation and quantitative PCR. Hematocrit was measured using a Microhematocrit EZ Reader (LM Scientific #ZCP-EZRD-HEM7).

vii. Object Recognition Memory Test:

Male BALB/c mice were treated with QPO (40 μg/kilogram, i.p.) or an equivalent injection volume of PBS (˜150 μL, i.p.) 1×/day for four days. Mice were then habituated to the 42×28×18 cm testing cages under experimental conditions (no bedding, 40-50 lux) for 1 hr. One day after habituation, mice were trained on two identical objects (either two 50 mL Falcon Tubes or two Lego brick towers of equivalent size) placed 6 cm from either end, and 10-12 cm from either side for 30 minutes, at which point mice were returned to their home cages. After 24 hours mice were placed in the testing cages containing their trained object and a novel object for 5 minutes. The test was recorded with a Basler acA 1300-60 gm NIR camera and scored for the duration of time spent exploring each object in the 5-minute testing period. Object exploration was determined by the length of time the mice spent with its nose pointed at and/or sniffing the object within 2 cm. Climbing behavior was not considered exploration. Data was expressed as a discrimination index determined by the amount of time spent exploring the novel and familiar objects (see Equation 1.1).

DI=t_(novel object)/((t_(novel object)+t_(familiar object))) Equation 1.1

viii. Forced Swim Test:

The forced swim test (FST) was carried out in two clear cylindrical plastic tubs (20 cm diameter, 25 cm height, 16 cm water height, 26-28° C.) placed in NIR chambers (180-200 lux/chamber). Male BALB/c mice were treated with QPO (40 ug/kg i.p.) or an equivalent injection volume of PBS (˜150 uL, i.p.) 5 hrs. before each 6-minute testing session. Each testing session was recorded using a Basler acA 1300-60 gm NIR camera and scored using automated tracking software (Ethovision XT 14, Noldus Information Technology, Leeburg Va. USA) for behavioral mobility and immobility during the final 4 minutes of the test.

ix. Open Field Test:

Mice were habituated to 40×40×50 cm black tubs in the testing room for 1 hour (250-300 lux in the center of the tub, 150-250 lux at the edges) the day before testing. On the test day, mice were placed in the center of the tubs and allowed to move freely for 10 minutes. The trials were recorded with a Basler acA 1300-60 gm NIR camera and were scored for total distance moved (in cm) using automated tracking software (Ethovision XT 14, Noldus Information Technology, Leeburg Va. USA).

x. Novelty Induced Hypophagia Test:

The Novelty Induced Hypophagia Test (NIHT) was performed as previously described by Sampath, McWhirt, Sathyanesan, and Newton (15). Over the next two days, testing proceeded in their home cage (45-50 lux), then in a novel cage (550-600 lux, no bedding). Testing periods were 30 minutes long and recorded using a Basler acA Color Camera. The duration of time between introduction of the sweetened condensed milk to the first drink was recorded as “latency to drink”.

xi. QPO Homology Model Design:

The homology model of the triple-substitution mutant QPO (K-20,45,97-Q) used for molecular dynamics simulations was based on the solved crystal structure of non-glycosylated EPO (ngEPO) bound to EPOR/EPOR (Protein Data Bank: 1EER, Chain A, Resolution 1.9 Å) using Molecular Operating Environment 2018 (MOE). All protein models were protonated within MOE (pH=7.4, T=310K) and refined to an RMS gradient of 0.1 kCal/mol/A before performing energy minimization to reduce torsional stress.

xii. Molecular Dynamics Simulations:

Molecular dynamics simulations were performed on the University of South Dakota's “Lawrence” Supercomputing Cluster using Molecular Operating Environment v2018.1 (MOE) in tandem with Nanoscale Molecular Dynamics (NAMD v2.13) for Linux Multicore software. QPO and ngEPO were placed into the binding pocket of the active conformation of EPOR/EPOR in the same position and orientation by superposition onto Chain 1 of the 1EER pdb file. After placement, the complex was energy minimized to a 0.1 kCal/mol/A threshold. All simulations were performed in explicit solvent (3D-RISM model, density: —1 g/mL) with the following parameters: time step: 0.002 ps, heat: 3 ns to T=310K, equilibration time: 6 ns, production time: 26 ns, sample rate: 5.0 ps, Langevin damping: 5/dT, pressure: 101.325 bar. Simulations were run until equilibrium was achieved (between 24 and 34 ns), at which point there were compiled and analyzed using the MOE Molecular Dynamics Analysis suite.

xiii. Free Energy of Binding (AGB) Estimation:

Post-convergence, representative “snapshots” of each simulation were taken to perform the necessary calculations for the estimation of the AGB for each active site of the bound ngEPO/QPO-EPOR/EPOR complex (N=4 measurements/trajectory, 4 trajectories/variant, at minimum 1 ns separation between each measurement). AGB and the associated dissociation constants (KD) were calculated as previously reported.

xiv. Statistical Measurement:

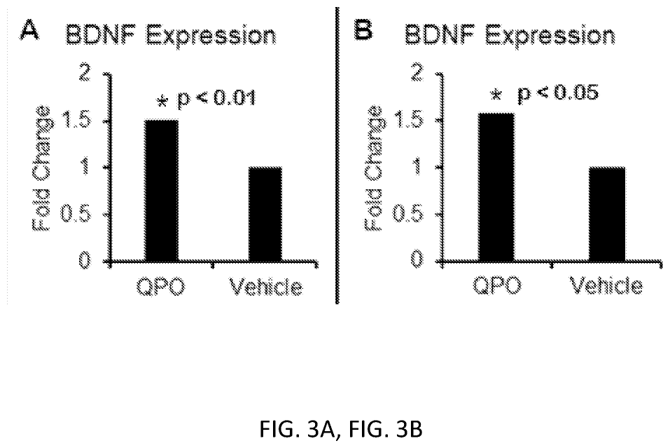

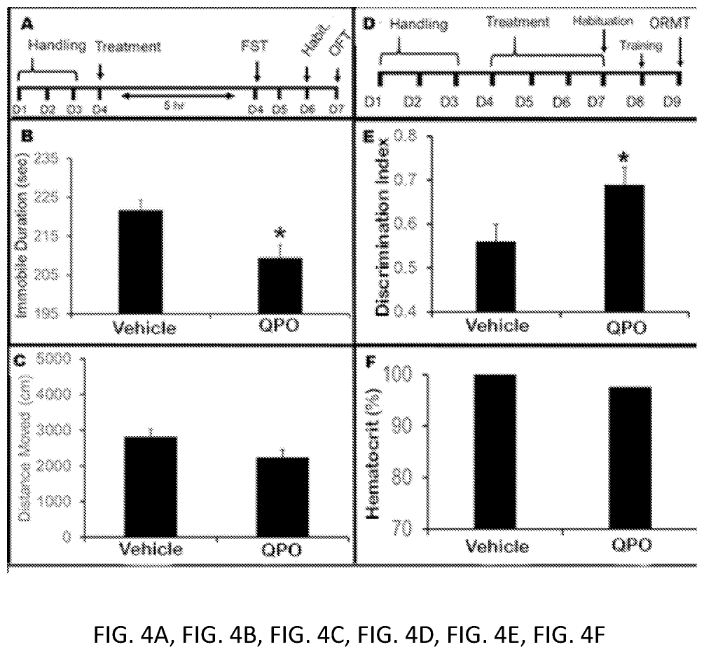

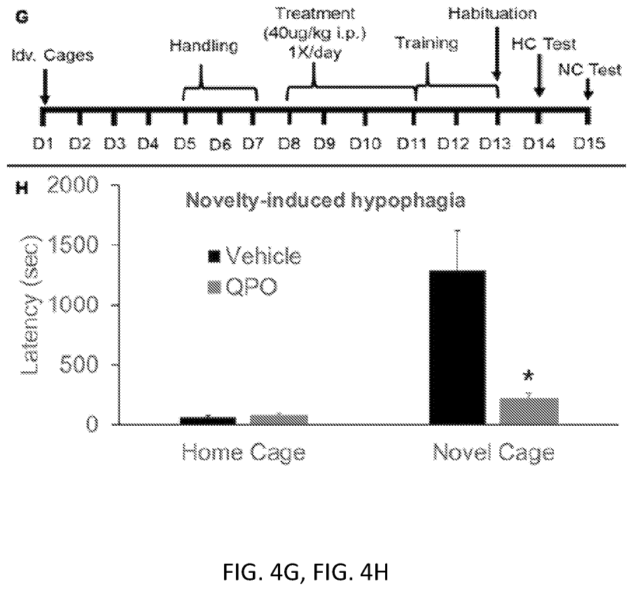

All data was presented as the mean+/−standard error of the mean. In FIGS. 3 and 4F, the error and statistics were calculated for the raw data (Δct for gene expression data, and raw hematocrit for FIG. 3), but were transformed mathematically into a more easily interpreted form (fold-change in expression calculated from ΔΔct and hematocrit % change compared to vehicle treated controls). The associated raw data can be found in supplementary tables ST1 and ST2 respectively. Any and all outliers were removed using Grubb's tests, and statistical significance for behavioral data and computational tests were determined using the student's T-test. A p-value of <0.05 was considered statistically significant. Statistical tests were performed using Microsoft Excel.

3. Results:

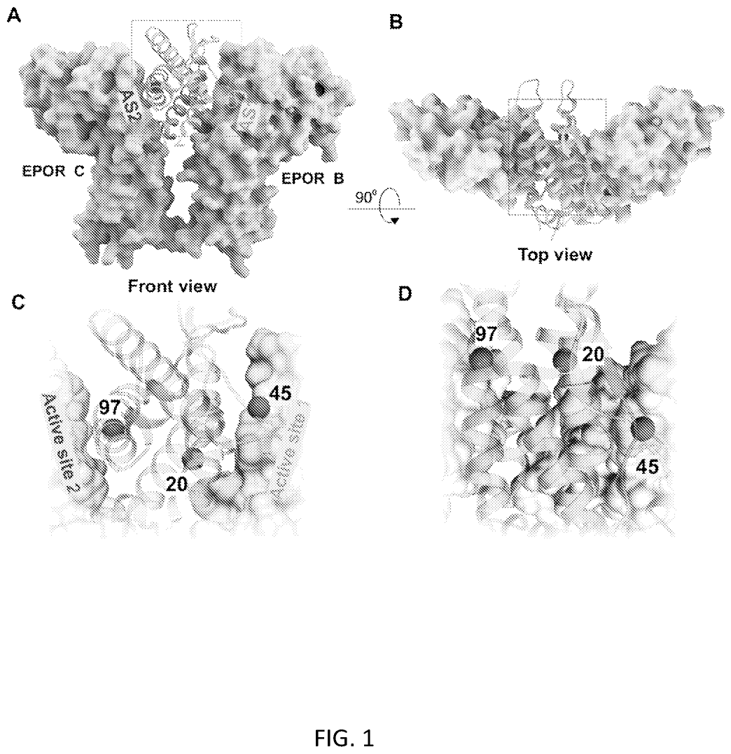

For orientation purposes, FIG. 1 displays the ribbon structure of the bound state of non-glycosylated erythropoietin (ngEPO, in green) to EPOR/EPOR (in red) from two perspectives, with AS1 and AS2 labeled. The three surface lysine residues changed to glutamine in QPO are shown in blue, using a space-filling model.

i. Purification and Characterization:

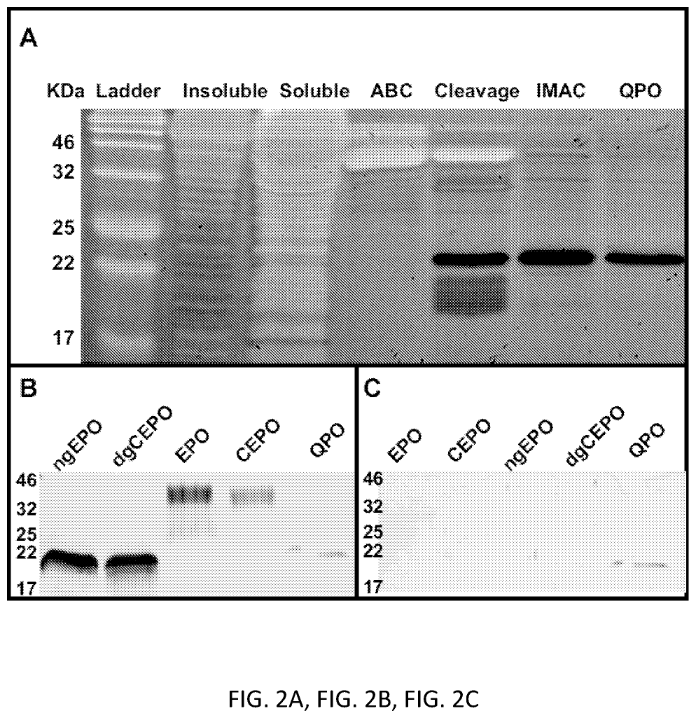

The purification procedure for QPO was verified via silver staining. In FIG. 2A, the protein isolated from each purification step (see Materials and Methods) is shown, ending with the final QPO-6×His product in 1×PBS. The isolated QPO was then characterized by western blot to determine its reactivity to Anti-EPO (FIG. 2B) and Anti-6×Histidine (FIG. 2C) antibodies, with comparisons to ngEPO, deglycosylated CEPO (dgCEPO), EPO, and CEPO as positive and negative controls, respectively.

ii. BDNF Gene Expression:

In FIG. 3A, it is shown that QPO treatment of neuronally differentiated PC-12 cells (100 ng/mL, 3 hr. incubation) caused an approximate 50% increase in BDNF expression when compared to vehicle treated controls (p<0.01, N=6). In-vivo the same upregulation of BDNF can be seen in the mouse hippocampus a full 48 hours after receiving the final dose of a 10 dose QPO treatment regimen (40 μg/kg i.p.) (p<0.05, N=6). All gene expression results are presented as fold change in expression compared to vehicle treated controls.

iii. Behavioral Assays and Hematopoiesis:

First, to screen for potential antidepressant-like activity, BALB/c mice treated with QPO (40 μg/kg, i.p.) were administered the FST. The treatment and testing schedule, and the resulting changes to immobility duration, can be seen in FIGS. 4A and 4B, respectively. QPO treated mice showed a significantly decreased immobility duration compared to vehicle treated controls (p<0.05, N=6). To prevent confounds arising from potential stimulant or depressant properties of the drug, the same cohort of mice were administered the OFT (FIG. 4C). As expected, there was no significant difference in total distance moved over the testing period, implying that the FST results reflect antidepressant-like effects (p>0.05, N=6). To test the effects of QPO on cognitive function, the ORMT was performed. The training and testing schedule, as well as the results, are presented in FIGS. 4D and 4E. QPO treated mice showed significantly increased preference for the novel object compared to vehicle treated controls (p<0.05, N=6 for QPO, 8 for vehicle). After a full 10 doses of the drug over the course of 2 weeks of behavioral testing, hematocrit was measured from truncal blood to determine QPO's in-vivo hematopoietic activity. As can be seen in FIG. 4F there is no significant difference in hematocrit % between QPO treated and vehicle treated mice (p>0.05, N=6). Lastly, to further confirm the antidepressant-like effects of QPO, the NIHT was performed. The treatment and testing schedule, and the results are in FIGS. 4G and 4H respectively. In the home cage, there was no significant difference between the latency-to-drink of QPO treated and vehicle treated mice (p>0.05, N=6). However, in the novel cage, QPO treated mice showed a significantly decreased latency-to-drink compared to vehicle treated controls, implying antidepressant activity (p<0.05, N=6).

FIGS. 29A and 29B are similar to FIGS. 4E and 4F except that FIG. 29 shows the results of cognitive function and enhancing memory recognition in an Alzheimer's disease mouse model (5×FAD) whereas FIG. 4 uses a wild type mouse. Similar results are seen. QPO treated mice rescues recognition memory deficits in 5×FAD mice. Five doses at 40 ug/kg/day of QPO rescued the deficit.

iv. Binding Affinity Assay:

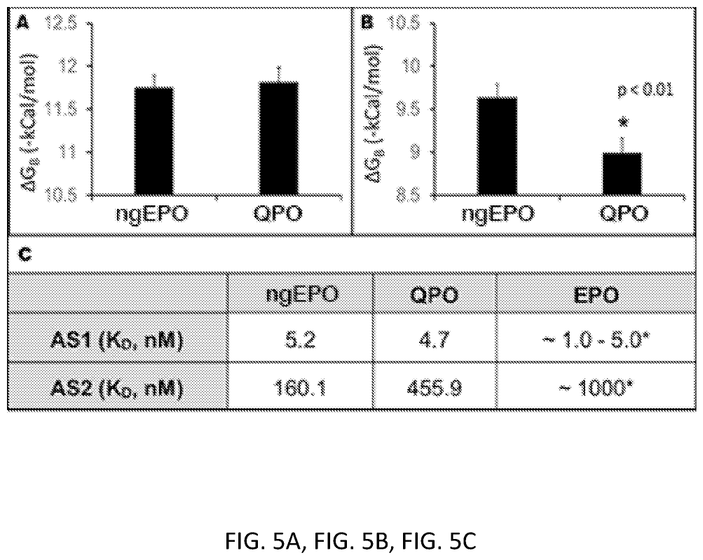

The in-silico binding affinity (AGB) of QPO and ngEPO to the EPOR/EPOR homodimer were calculated for each active site. The AS1 values for both are shown in FIG. 5A, while the AS2 values are shown in FIG. 5B. QPO showed significantly decreased AGB at AS2 compared to ngEPO, but not at AS1 (p<0.05, N=16). These AGB were used to calculate the AS1 and AS2 dissociation constants for QPO and ngEPO and compared them to known experimental constants for fully glycosylated EPO (see FIG. 5C). QPO and ngEPO both show AS1 binding affinities that are similar to that of EPO, while ngEPO shows increased binding affinity to AS2 compared to EPO.

### 4. Discussion

i. Evaluation of QPO's Antidepressant-Like Activity:

To determine if QPO is a successful mimetic of CEPO, it is important to assess its effects on cognition, mood, and neurotrophic gene regulation.

First, in terms of cognitive effects the ORMT has been previously shown to be highly specific to the perirhinal and hippocampal regions of the brain, where a positive result (increased preference for the novel object) has been associated with increased cognitive function. QPO treatment at doses equivalent to those used for CEPO administration result in a significantly increased preference for the novel object.

Second, the results of the FST and NIHT (reduced immobility duration and decreased latency-to-drink in the novel cage, respectively) imply that QPO has functional antidepressant-like activity. Like CEPO, treatment with QPO caused significant upregulation in BDNF expression in-vitro and in-vivo, which has previously been demonstrated as one of the more promising neurotrophic factors associated with the symptomatic improvement of depressive behavior in animal models.

Lastly, QPO is completely nonhematopoietic in-vivo while retaining neuroactivity, which when coupled with the behavioral data, signaling data, and the substituted residues' with similar physiochemical properties to homocitrulline, it can be argued that QPO is a successful mimetic of CEPO and mediates its effects through the same neurotrophic mechanism.

ii. Effect of Glycosylation and K-20,45,90-Q Mutation on EPOR/EPOR Active Site Affinity:

Tsuda et al. showed that as the glycosylation level of EPO decreases, the affinity of EPO for the EPOR/EPOR homodimer receptor increases. Surprisingly, molecular dynamics simulations of ngEPO revealed that complete removal of EPO's glycan groups results not in an overall receptor affinity increase, but rather in a selective increase in AS2 affinity compared to the glycosylated form's experimental values. This may mean that the glycosylation sites of EPO serve to sterically hinder binding to AS2, and only AS2. When comparing the binding strength of QPO vs ngEPO to EPOR/EPOR, the data suggest that the substitution of K-20,45,97-Q causes significantly decreased binding at only AS2, with AS1's affinity being largely unaffected. This implies that the K20 and K45 residues of EPO are nonessential for receptor binding at AS1, but that K97 has a significant impact on binding affinity to AS2. As QPO mimics CEPOs physiochemical properties (especially with regards to the homodimer active sites), it can be inferred that the glycosylation of CEPO, when coupled with the neutral homocitrulline at position 97 would serve to decrease AS2 affinity still further, which in turn provides a clue to the structure of the theoretical IRR.

iii. On the Structure of the Innate Repair Receptor

As of writing, though there is strong evidence associating IRR activity with EPO's tissue protective effects, there is little evidence establishing that the IRR mediates EPO's behavioral effects in the CNS. The revelation that QPO, and by extension CEPO, binds AS1 as strongly as EPO provides strong evidence that at least one half of the IRR complex involves EPO bound to AS1 of an EPOR monomer. In addition, it also implies that the residues of EPO associated with AS2 are nonessential to binding to the IRR and are unlikely to be involved in the CD131 active site(s).

iv. Conclusion:

In neuronally differentiated PC-12 cells and in the hippocampi of treated mice, QPO upregulated the expression of BDNF, a neurotrophic factor known for its antidepressant properties. In behavioral studies, treatment with QPO showed beneficial cognitive and antidepressant-like effects in BALB/c mice in the ORMT, FST, and NIHT. The computational binding assays for ngEPO and QPO, in conjunction with the signaling data, provide evidence that AS2 binding is unnecessary for the activation of neurotrophic signaling by EPO through the IRR. Lastly, QPO showed behavioral and signaling activity similar to CEPO, implying that the production of a polypeptide mimetic of CEPO was successful, and therefore has high potential as a possible psychiatric pharmacotherapy.

### B. QPO and RPO Design

Constructs were prepared comprising recombinant EPO polypeptides. One recombinant polypeptide (QPO) has K to Q substitutions at positions 20, 45 and 97 of wild type human Epo. One recombinant polypeptide (RPO) has K to R substitutions at positions 20, 45 and 97 of wild type human Epo.

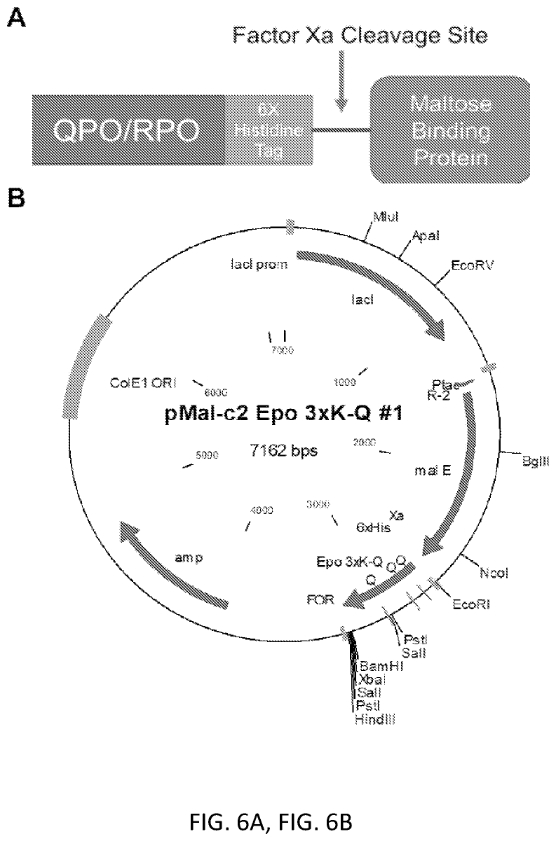

The QPO and RPO were expressed as a fusion protein with MBP in E. coli (FIG. 6). These constructs showed improved solubility, improved yield, two-step purification, decreased expense, easier expression, easier cultures, and easily scaled compared to other recombinant EPOs.

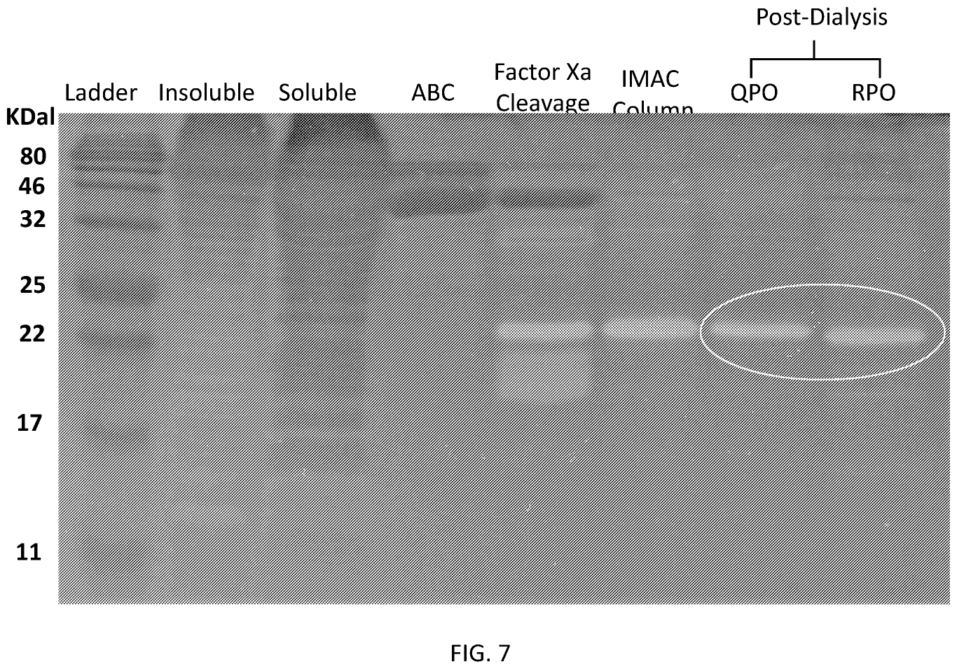

FIG. 7 shows the purification of both QPO and RPO.

In vitro (rat pheochromocytoma 12 (PC-12) cells) and in vivo (Balb/c mice) experimental models were used. PC-12 cells are commonly used to study neuronal gene regulation after EPO or EPO variant treatments. Balb/c mice are highly stress sensitive and show strong reactions to antidepressant medications.

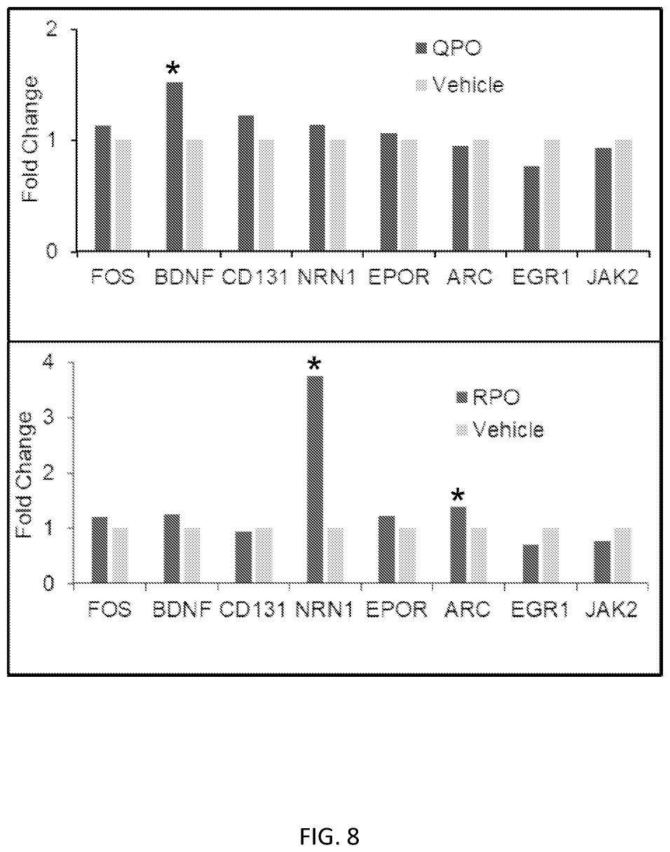

FIG. 8 shows PC-12 cell gene profiles. QPO treatment resulted in upregulation of BDNF (p<0.001). RPO treatment resulted in upregulation of NRN1 (p<0.05) and upregulation of ARC (p<0.05).

The following behavioral assays were used: object recognition memory test (ORMT), forced swim test (FST), open field test (OFT), and novelty induced hyophagia test (NIHT). The ORMT is a test of cognitive function. The FST is a screening test for antidepressant activity. The OFT is a test of general locomotor activity. The NIHT is a test of chronic antidepressant activity.

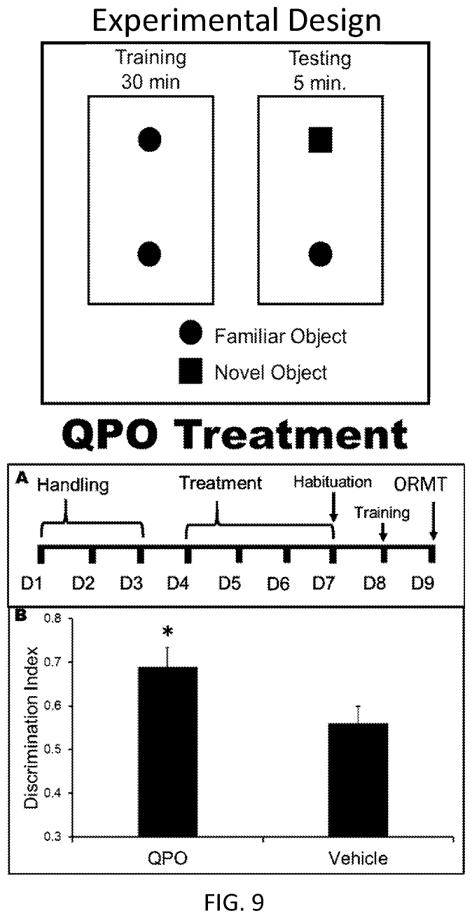

FIG. 9 shows the results of an ORMT. The QPO treatment shows a positive ORMT test.

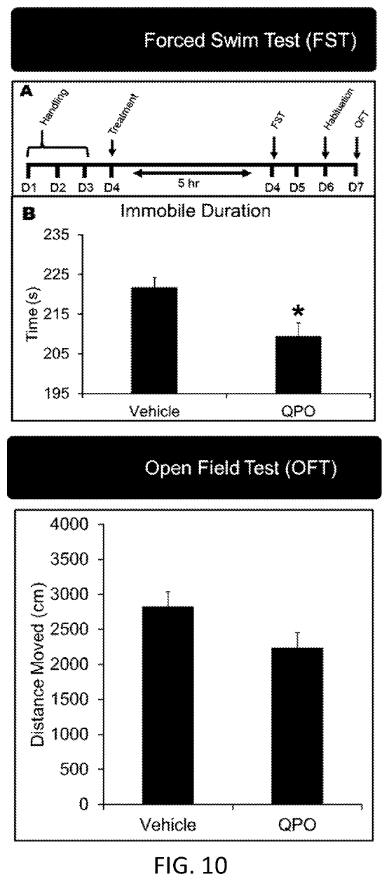

FIG. 10 shows the results of a FST. Mice were placed in water (ranging from 26-27° C.) for 6 minutes. Mice were scored for the duration of time spent mobile or immobile. Antidepressant activity is determined by more time spent mobile and less time spent immobile. The QPO treatment shows a positive FST test.

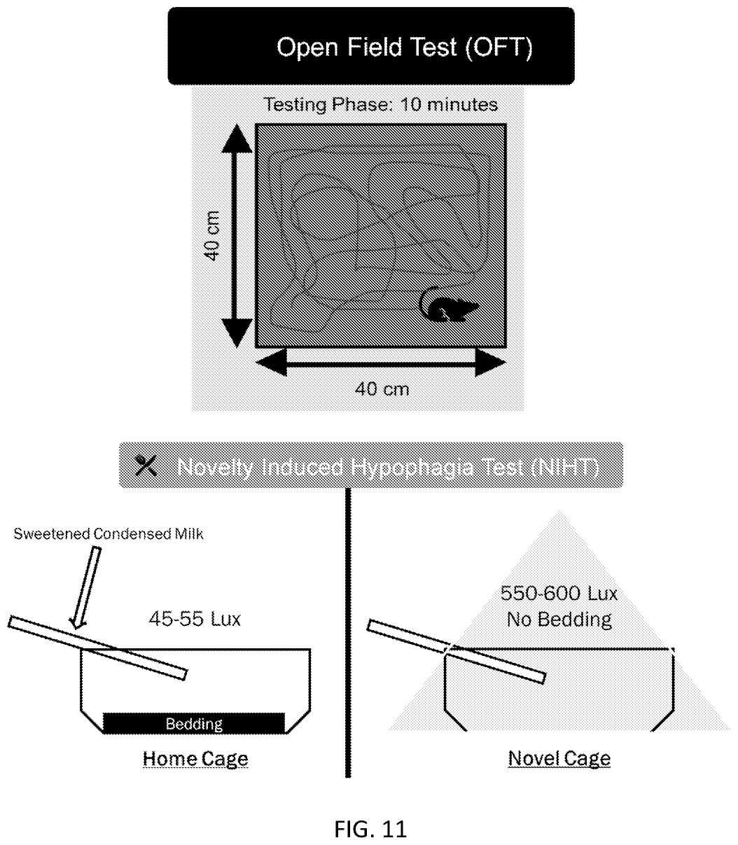

FIG. 11 shows the set up for a NIHT.

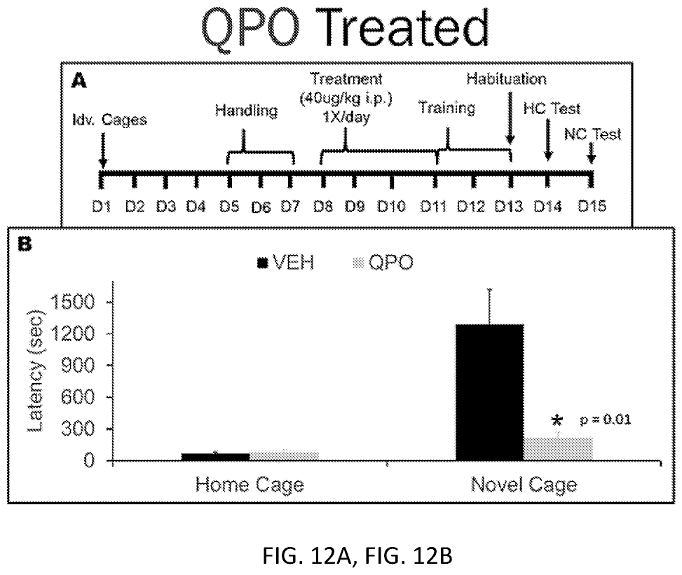

FIG. 12 shows the results of the NIHT study. The QPO treatment shows a positive NIHT test.

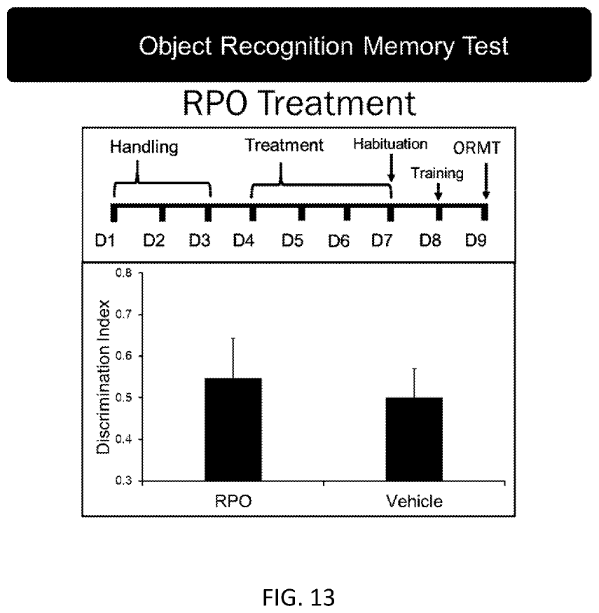

FIG. 13 shows the results of an ORMT. The RPO treatment shows a negative ORMT test. There is not a significant difference between the vehicle control and RPO treatment.

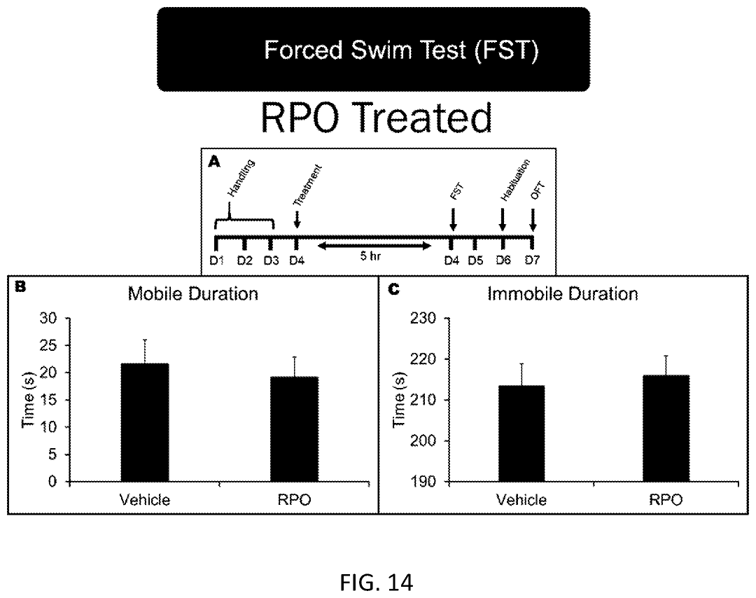

FIG. 14 shows the results of a FST in RPO treated mice. These results indicate that RPO does not possess antidepressant-like activity.

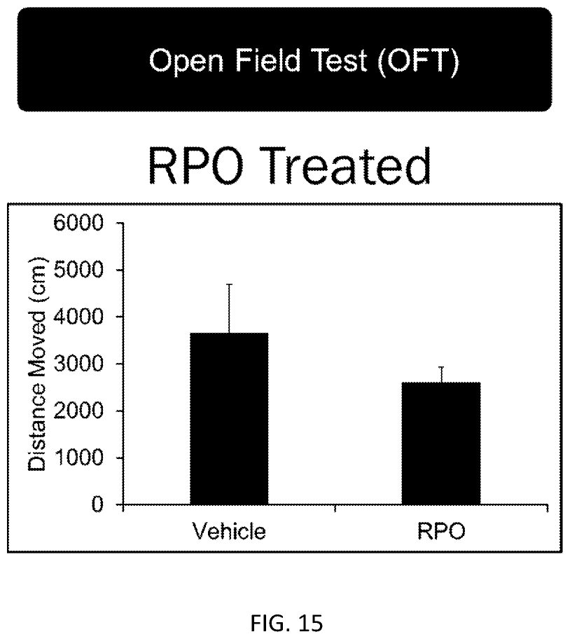

FIG. 15 shows the results of an OFT. RPO shows no antidepressant or pro-cognitive effects in vivo.

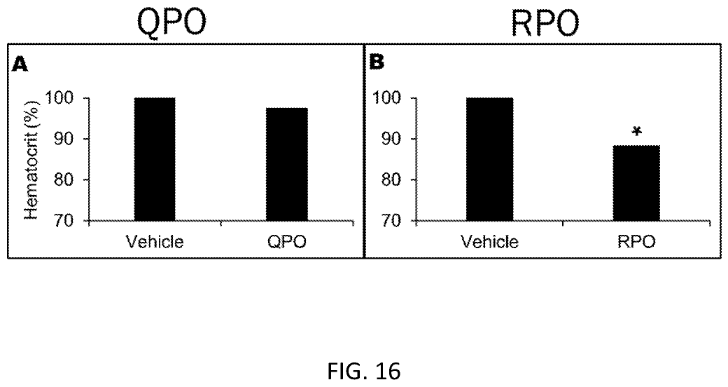

FIG. 16 provides the non-hematopoietic effect. Hematocrit was measured after 10 doses of either QPO, RPO, or control. Treatment with QPO causes no significant change in hematocrit vs vehicle treated controls. Treatment with RPO causes a ˜10% decrease (p<0.01) in hematocrit. RPO interferes with endogenous hematopoietic signaling.

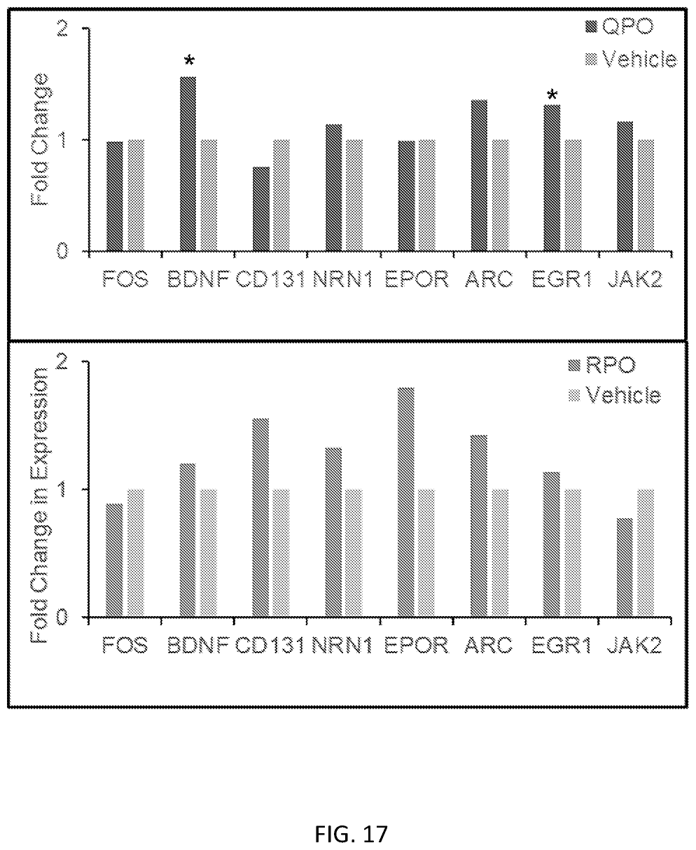

FIG. 17 shows the hippocampal gene profiles when treating with either QPO or RPO. QPO Treatment resulted in an upregulation of BDNF (p<0.05) and an upregulation of EGR1 (p<0.05). RPO Treatment resulted in EPOR trending (p=0.09). Thus, QPO upregulates BDNF in vivo and RPO does not upregulate neurotrophic factors in vivo.

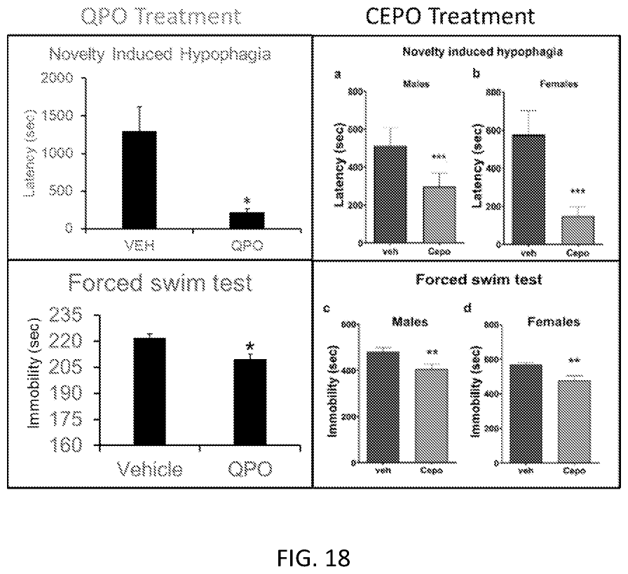

FIG. 18 shows that QPO has similar antidepressant effects to CEPO.

### C. Characterization of Trophic Factor Activity in the Brain

This study is designed for the characterization of trophic factor activity in the brain. The central role played by neurotrophic factors in modulating neuronal function and behavioral response is well known. Furthermore, preclinical and clinical studies have strongly implicated neurotrophic signaling deficits in the pathophysiology of depression. Numerous animal model studies have demonstrated the robust antidepressant-like effects of neurotrophic factors. Despite emerging as a valid molecular target for therapeutic development, clinical translation has not yet occurred. With the exception of erythropoietin (EPO), most neurotrophic factors do not cross the blood-brain barrier and require direct brain delivery. Preclinical animal model studies, human testing, and patient population clinical trials have shown that EPO is a promising neurotrophic factor for the treatment of psychiatric disorders. However, the clinical use of EPO as a CNS drug is significantly limited by its inherent erythropoietic activity that has repeatedly produced dangerous blood count elevation and hematological complications in clinical trials.

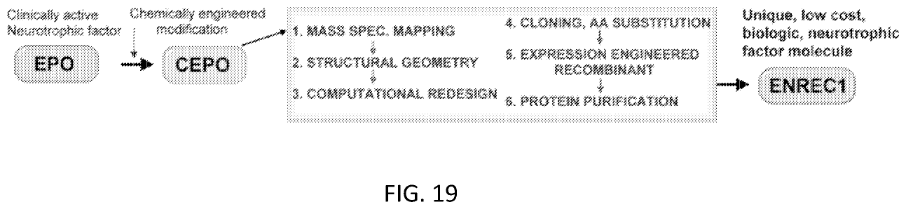

Carbamylated EPO (CEPO), a chemically engineered structural modification, renders EPO non-erythropoietic while preserving its neurotrophic activity. CEPO is therefore a molecule of substantial value for obtaining mechanistic insight into the erythropoiesis-independent, molecular signaling and behavioral actions of EPO. However, CEPO remains expensive to produce as it requires high purity mammalian EPO as the starting material and also requires careful chemical engineering of each production batch to alter its structure and render it non-erythropoietic. In order to safely harness and develop the promising results obtained with Phase II EPO trials a genetic version of CEPO was produced by employing a structural biology approach. This engineered recombinant (Enrecl) was computationally designed using CEPO as the template (see FIG. 19). Enrecl was produced in a low-cost bacterial expression system without the large carbohydrate/sugar moiety that is carried by both EPO and CEPO. The antidepressant-like activity of EPO can be reproduced by a strategically engineered non-erythropoietic bacterial recombinant protein. The major goals of this study are: 1) test whether Enrecl can produce antidepressant-like effects in established rodent behavioral models and 2) examine the mechanistic basis of Enrecl activity in the hippocampus.

1. Determine the Antidepressant Efficacy of Enrecl

Using a combination of protein chemistry, structural biology and protein engineering methodologies a structurally simplified neurotrophic molecule has been produced that mimics CEPOs molecular profile. The protein expression scheme employed and optimized to produce Enrecl in the high μg range can be proportionally scaled-up to obtain yields in the mg range. The antidepressant efficacy of Enrecl can be tested in a set of established mouse behavioral paradigms, including the forced swim test (FST), tail suspension test (TST) and novelty-induced hypophagia (NIH). Consistent behavioral effects across this battery of tests of antidepressant efficacy can provide confidence that Enrecl can produce similar antidepressant effects to EPO.

2. Determine the Hippocampal Neurogenic and Neurotrophic Potential of Enrecl

Preclinical and clinical studies have implicated the hippocampus and shown that EPO produces both faster (<1 week) and slower onset behavioral effects (>2 weeks). The mechanistic basis for this is unknown. Dentate gyrus (DG) neurotrophin induction can be involved in the faster effects and neurogenic activity in the subgranular zone (SGZ) for the slower onset effects. Functionally similar effects can be seen with EnRec1. The regulation of Ascl1, a neurogenic potential marker gene, can be examined in the SGZ and the EPO-induced neurotrophins, BDNF, VGF and neuritin can be tested in the DG. Laser microdissection can be used to determine hippocampal cell layer-specific, EnRec1-induced gene regulation.

The proposal addresses major limitations of a current and clinically relevant neurotrophic approach to treat psychiatric disorders. The neurotrophic molecule, produced by progressing through six developmental stages, overcomes key issues pertaining to erythropoiesis, cost, and manufacturing reproducibility. Moreover, the structure-function principles that emerge can be extended to manipulate structurally similar proteins to obtain molecules with unique therapeutic functionality. This strategy can lead to a new paradigm in psychiatric drug development.

3. Significance

The translation of laboratory findings into successful clinical therapies has been particularly slow in CNS disorders. Promising psychiatric disease targets identified in preclinical research have rarely succeeded in clinical testing. No new breakthrough drugs have been developed for depression and schizophrenia in the last 20 and 50 years respectively (1). The high failure rate and the rising cost of new drug development, estimated at $1.8 billion (2), has forced pharmaceutical companies to downsize neuroscience research. A count of 11 highly visible pharma CNS investments shows that more than half the programs have been terminated over the past 5 years (3). Continuation of this trend could create a crisis in drug development for psychiatric diseases that are desperately in need of new therapies (4). In order to address the double-edged challenge of escalating developmental costs and lack of efficacy there is currently an effort to repurpose existing candidates for new therapeutic uses. Ideal targets would match current disease hypotheses with therapeutically desired molecular function. This strategy involves careful examination of targets that have progressed to safe clinical testing and produced pharmacological efficacy in humans.

i. Neurotrophic Therapy in Psychiatric Disorders

The multiple levels at which trophic signaling could influence behavioral deficits has been increasingly understood from a large body of evidence including postmortem, live imaging and pre-clinical animal model studies, and led to the formulation of a cellular and molecular hypothesis of depression/antidepressant action. Neuronal atrophy caused by dysregulation of neurotrophic signaling underlies the behavioral consequences of depressive disorders. Although the neurotrophic hypothesis has been validated by numerous reports in pre-clinical models, the robust findings have not led to successful clinical translation. A variety of small molecules and biological neurotrophic factors have been tested in cell lines, preclinical and clinical studies. The major drawbacks with small molecules have been pleiotropic and off target effects. Invasive delivery, adverse systemic effects and lack of efficacy due to inadequate brain penetration precluded further clinical advancement in the case of biomolecules. A therapeutically approved molecule with a strong clinical profile and established CNS neurotrophic activity would satisfy essential requirements to test the utility of the neurotrophic approach in the treatment of psychiatric disorders. In recent years, erythropoietin (EPO) has emerged as a promising agent because it has a long track record of clinical use, is transported across the blood brain barrier and produces robust neurotrophic actions. Although primarily investigated and highly prescribed world-wide for its ability to cure anemia, numerous preclinical studies (>360) have demonstrated its robust neurotrophic and cytoprotective effects in multiple CNS disease models. These properties swiftly propelled it into clinical trials for schizophrenia, depression and neurocognition.

ii. Engineering a Recombinant Neurotrophin

Despite promising results that validated the clinical applicability of the neurotrophic approach, EPO clinical trials revealed that the erythropoietic activity of EPO produces adverse elevation in blood counts and blood viscosity upon chronic administration. These hematological consequences also increased mortality in a large EPO stroke trial. In order to overcome the erythropoietic side effects and successfully reposition EPO for use in CNS disorders, research efforts have focused on non-erythropoietic derivatives. Non-erythropoietic peptides derived from the crystal structure of EPO produced desirable results in preclinical studies but are rapidly degraded and require administration at 10× the concentration of EPO. Enzymatic modification of the carbohydrate content (AsialoEPO) is an alternate approach to reduce erythropoietic activity by enhancing renal clearance. A chemically engineered non-erythropoietic EPO derivative, carbamylated EPO (CEPO), is an attractive and innovative molecule for development as it structurally alters the protein to specifically preclude the erythropoietic signaling pathway but fully retains neurotrophic activity. However, all EPO modifications that rely on mammalian cell lines are unlikely to develop into mainstream therapies due to high production costs. Furthermore, the carbohydrate moiety is complex and can consist of 100s of variants on the same amino acid sequence. It is therefore highly desirable to develop a simplified neurotrophic molecule that encodes CEPO's structure at the DNA level by specific amino acid substitutions. This neurotrophin can then be reproducibly generated at any scale using a cost effective bacterial system.

4. Innovation

1) Conceptual: All current prescriptions to treat psychiatric disorders are serendipitously discovered small molecules based upon the neurotransmitter hypothesis of disease.

2) Drug template development: The strategy to obtain critical atom-level details from a chemically engineered modification and utilize it in developing a simplified candidate represents the biological equivalent of combinatorial synthesis and a new direction in psychiatric drug development. The atomic geometry of the chemical modification was converted into equivalent and manageable amino acid substitutions so that it could be encoded in the DNA. This strategy can be extended to structurally similar trophic proteins.

3) Choice of candidate molecule: The clinical safety profile of EPO is outstanding and is used by millions worldwide. It is therefore unlikely to fail toxicity tests, which is a major concern with small molecules. Its mechanism of action in curing anemia is well understood. The target receptor is well characterized and high resolution crystal structure is available. The dangerous increase in RBC counts and other hematological changes occur only when it is abused (in endurance sports) or chronically administered to non-anemic individuals. It is an endogenous molecule with high affinity for its receptor. This greatly reduces the possibility of off target effects.

4) Translational: Production costs are an important aspect of new technological advances. To this end multiple methods were tested to express functionally active recombinant protein. Furthermore, molecular tags were included to simplify protein purification in a 2 step process. This protein expression methodology can be critical when scaling up production as it improves protein recovery and greatly reduces contamination with bacterial toxin proteins.

5. Approach

The goal is to obtain detailed resolution of a promising, clinically viable neurotrophic factor and utilize the information to strategically manipulate it by employing a computational structural biology approach. The validity of the strategy is tested by producing the full length redesigned protein in a bacterial expression system. The intent in fully progressing through all the steps involved in the production of active protein is to demonstrate the feasibility of employing a computational approach to perform a targeted alteration of an existing protein and obtain new functionality. As the active molecule is in hand no developmental structural biology work is needed. The proposed experiments are focused on testing the in vivo functional efficacy of the neurotrophic recombinant, EnRec1, by examining brain region specific gene regulation and behavioral response in established antidepressant-responsive rodent paradigms. The experiments can be performed in male and female C57B16 mice.

Dosage: Erythropoietic activity is undetectable in EnRec1 based on blood parameters and cannot be dosed in conventional EPO units. Therefore the dosing regimen employed in CEPO studies will be employed, 40 μg/kg. A dose response analysis can be performed in the range of 4-40 μg/kg to determine the minimum effective dose in antidepressant behavioral assays. EnRec1 is stable in PBS (vehicle) and can be administered once daily (i. p.) for 5 days. As EPO produces SSRI-like behavioral actions in rodents and humans, fluoxetine can be used as the positive control.

Design and data analysis: The study design is a randomized control protocol with two main effects: Sex (Male vs Female) X Drug (EnRec1, Fluoxetine, and Vehicle). The prevalence of depression in women is significantly higher than in men with a (F: M) risk ratio of 2:1. It is therefore appropriate to test the antidepressant activity of a biologic in both sexes. As 3 behavioral tests (FST, NIH and TST) can be employed, this provides a 2×3×3 design and can adhere to the core principles outlined by NIH. FST and TST have predictive validity for antidepressant efficacy while NIH is responsive to chronic antidepressant administration. SAS can be used to generate uniformly-distributed random assignments. Data can be screened for outliers and non-normality. Outliers can be removed and non-normal response distributions can be transformed into relative normality. The primary analyses can include factorial ANOVAs. Post-hoc analyses can employ the Ryan-Einot-Gabriel-Welsch procedure to test for differences between means. To avoid alpha inflation, the overall alpha can be adjusted from 0.05 to 0.017 using a Bonferroni correction. an approximate power equal to 0.80 can be achieved for the main effects. Sample size was estimated using G*Power Version 3 for a fixed omnibus ANOVA with three between-groups effects and interactions. Statistical tests and analyses can be performed in consultation with a biostatistician.

6. Determine the Antidepressant Efficacy of Enrecl

Rationale and Studies: Results have been obtained from the clinic demonstrating the efficacy of EPO in treating major depression and neurocognitive deficits. However, further advancement is hindered by the elevation in RBC levels. CEPO is an ideal alternative as it is non-erythropoietic. Studies were conducted to understand the atomic configuration of CEPO and identified critical residues to substitute. This data was used to generate EnRec1 using a bacterial expression system.

i. Mapping Carbamylated (Cb) Amino Acids in CEPO

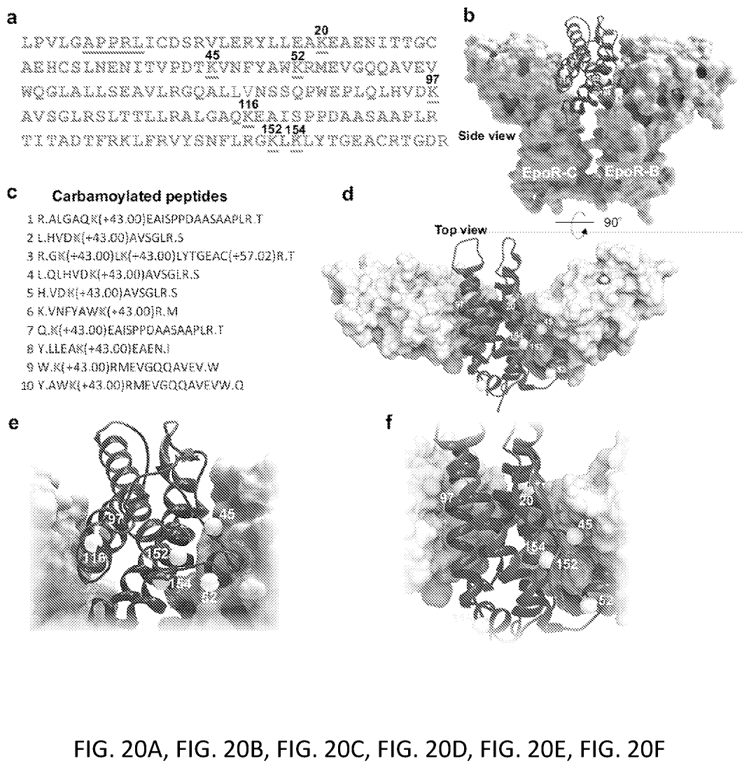

CEPO was produced by chemical modification (carbamylation) of pure EPO (Prospec Bio, specific activity of 120,000 IU/mg) by optimizing published protocols. As a first step towards the structural characterization of CEPO exhaustive mapping of >200 CEPO peptides was performed by mass spectrometry to obtain complete coverage of the protein and conclusively identified all Cb-residues (FIG. 20a). The spatial location and receptor proximity of the Cb-residues were then determined using the crystal structure of EPO bound to EPOR (Protein Data Base, PDB, 1EER), (FIG. 20, b-e). This analysis revealed that 3 lysine residues (crimson spheres, FIG. 20 c-e) were the ones most likely to influence CEPOs interaction with the receptor due to their proximity

ii. Interaction of Key Carbamylated with Receptor Active Site Amino Acids

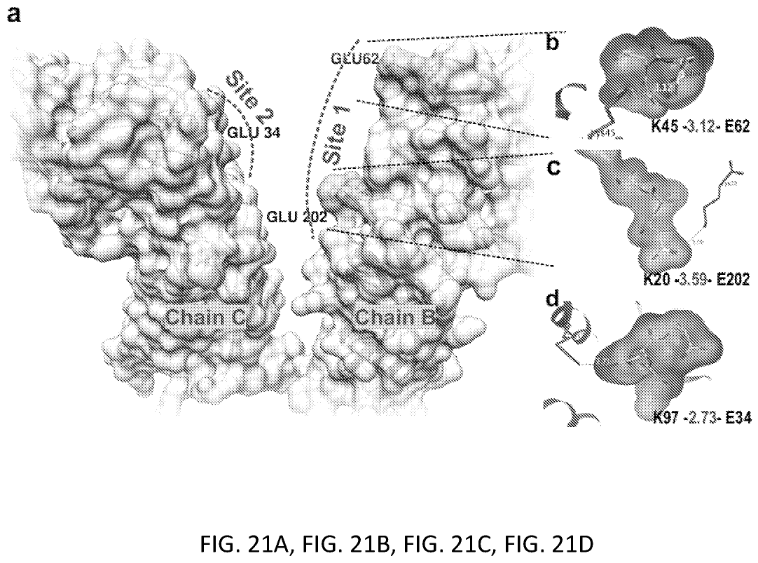

Precisely how close the Cb-residues, K45, K20 and K97, were to interacting receptor active site residues were determined. Atomic distance measurements were made using the Molecular Operating Environment (MOE) software package. Distances greater than 4 Å are unlikely to be involved in meaningful interactions. It was found that 3 acidic residues, two in active site 1 and one in active site 2 (E-glutamic acid, 34, 62 and 202, FIG. 21 a) that were within a 2.7 to 3.5 Å range from the basic K45, 20 and 97 of CEPO (FIG. 21 b-d) and therefore likely involved in regulating ligand-receptor interactions. Carbamylation of these lysine residues would be expected to alter the interactions.

iii. Carbamylation-Induced Alteration in Ligand-Receptor Interactions

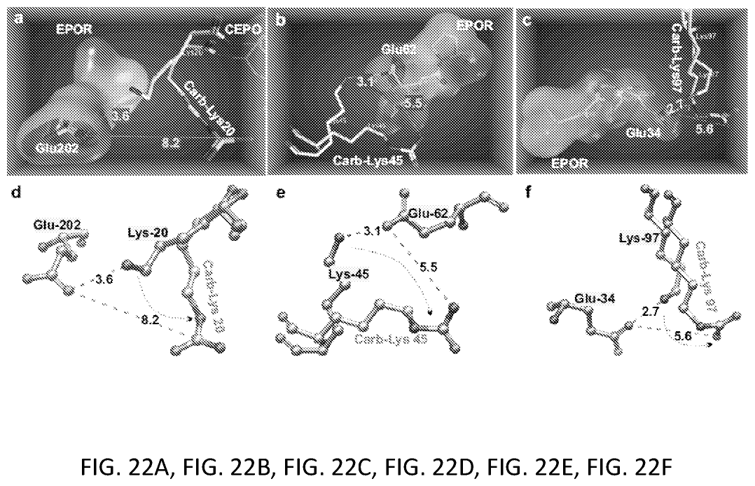

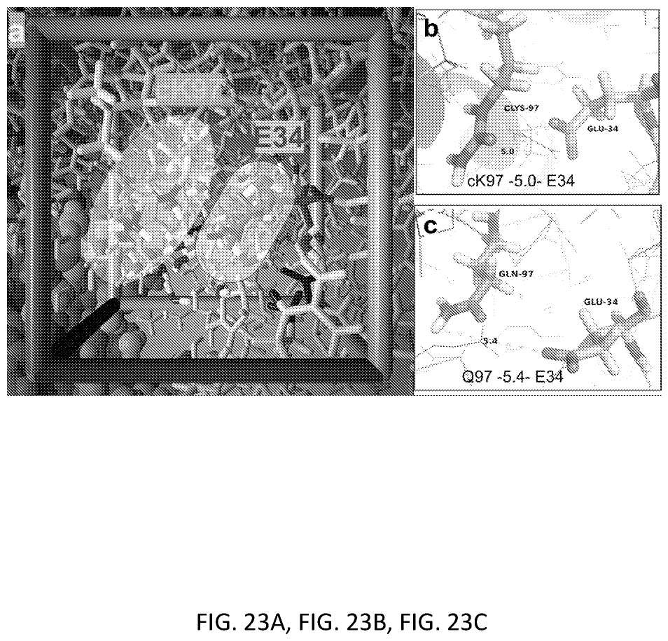

Carbamylation influenced interactions between Cb-lysines and receptor active site glutamic acid residues were studied. The MS studies had shown that Cb adds 4 new atoms, (C, N, H and O=43 Da) to each modified residue. The Cb modification were built onto K20, 45 and 97 in Chimera (UCSF); Protein preparation and optimization in Schrodinger. Molecular Dynamics (MD) simulations were performed using Yasara Structure software to gain insight into the structural, energetic, conformational and dynamic properties of carbamylation. A representative simulation cell (with reduced water) is shown in FIG. 23. Multiple force fields were tested, including AMBER, YASARA and YAMBER. The MD analysis revealed that Cb negates the salt bridges that exist between basic lysine and acidic glutamic acid, causing Cb-lysines to bend/move away from receptor glutamic acid residues (FIG. 22 a-c). For example, K97 which was 2.7 Å from E34 moves out of range to 5.6 Å (FIG. 22 c, f). A similar trend was noted in the case of K45 and K20. These studies indicate that Cb causes modified lysine residue side chains to flip away from the original position and cause a slight shift in ligand-receptor angular position because of the spatial distribution of these residues in the protein and the asymmetrical nature of binding the receptor.

iv. Computational Mutagenesis to Substitute Cb-Lysine

Based on the results of MD simulations the 3 lysine residues were substituted with glutamine in silico using PyMol and repeated the MD simulations in Yasara. The results in FIG. 23 show that glutamine reproduces the behavior of Cb-lysine. These studies indicated that a strategic substitution of Cb-lysine residues at 3 positions would be sufficient to render EPO structurally similar to CEPO. The MS mapping and computational biology yielded critical information that were used to generate a recombinant CEPO-like neurotrophin. Additional structural studies were performed to validate the model (Ramachandran plot, aggregate potential etc) and also examined the precise location of EPO glycans and found that the location of sugars does not influence receptor interaction. An active recombinant can be produced in a bacterial system incapable of glycosylation.

v. Expression Engineering EnRec1

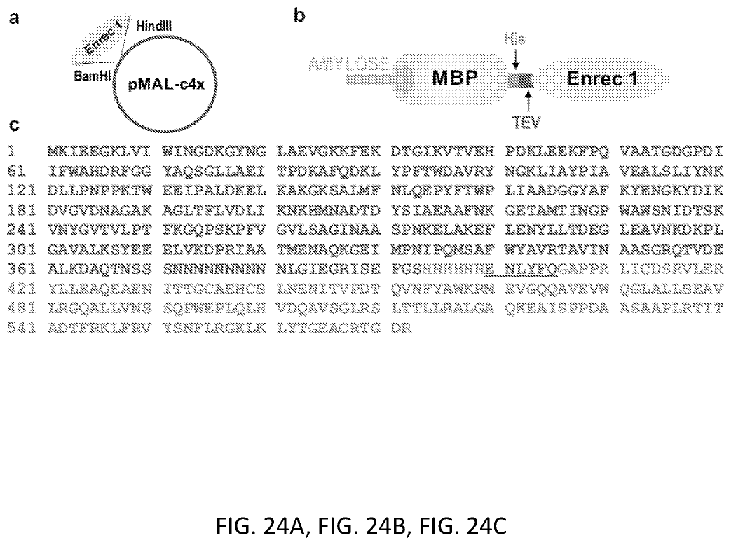

Obtaining soluble, functionally active mammalian proteins in a bacterial system is challenging. Although E. coli provide high yields and is 5×cheaper than mammalian expression systems, it lacks the machinery to fold complex proteins. Crystallographers had previously shown that the maltose binding protein (MBP) was highly effective in promoting the solubility and activity of “difficult to express” proteins. Furthermore, the approach was used recently to produce erythropoietic EPO. The EPO gene was cloned into the pMAL-c4×vector (NEB) and included specific cleavage (TEV) and purification (His) tags (FIG. 24 b, c) that would facilitate detection and purification. Amino acid substitutions are shown in red (24c). The plasmid was expressed in E. coli BL21 (DE3).

vi. Purification of EnRec1

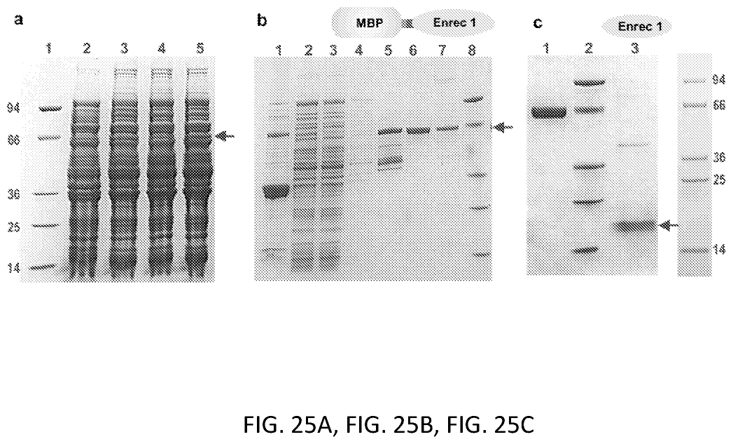

After testing several pilot expressions (80 ml) and gel analysis to determine the optimal conditions for yield (FIG. 25a) the expression was scaled to 1 L TB medium. Cells were harvested by centrifugation and disrupted by sonication. Soluble and insoluble fractions were partitioned and then processed using MBP trap columns (FIG. 25b). Pure fusion protein without any breakdown products was obtained (25b-lane 6). MBP was then cleaved using TEV protease to yield EnRec1 (25c, lane 3). Note that molecular weight of EnRec1 (18 kD) is substantially smaller than EPO (˜32 kD) due to exclusion of the large sugar moiety. This purification scheme enabled production of several hundred micrograms of EnRec1 which was utilized to test functional activity in cell lines and mice. EnRec1 is devoid of erythropoietic activity and also reproduced CEPOs gene profile. These studies demonstrate the ability to translate the results of structural biology analyses into the production of a new protein

Experiments EnRec1 protein production can be scaled up to facilitate behavioral testing by inoculating 4×2 L cultures in a large capacity shaking incubator. The identical scheme that was used to generate data (detailed above) can be employed without any modifications except for the use of higher capacity MBP-trap columns. Protein purity can be determined by silver staining of SDS-PAGE gels and amino acid substitution will be verified by MS analysis.

Behavioral response: Three established antidepressant assays, FST, NIH and TST that have been previously used to investigate EPO and CEPO's behavioral response can be employed. Animal treatments and behavioral analyses can be performed using protocols optimized for EPO and CEPO.

7. Determine the Hippocampal Neurogenic and Neurotrophic Potential of Enrecl

Rationale and Preliminary Studies: The hippocampal neurogenic and neurotrophic activity of EPO has been implicated in its behavioral effects by preclinical work. Clinical studies also suggest it as the potential mechanism of action. In addition to similarly elevating neurotrophic factors, cell signaling analysis of CEPO in neurosphere cultures has shown the induction of Ascl1/Mash1. Recently, Ascl1 has emerged as a critical transcription factor for adult hippocampal neurogenesis, integrating extracellular signals into a gene program that activates adult neural stem cells. Studies have been conducted in neuronal cell lines and mice to examine EnRec1-induced gene regulation.

i. EnRec1 Gene Profile in Neuronal Cell Line

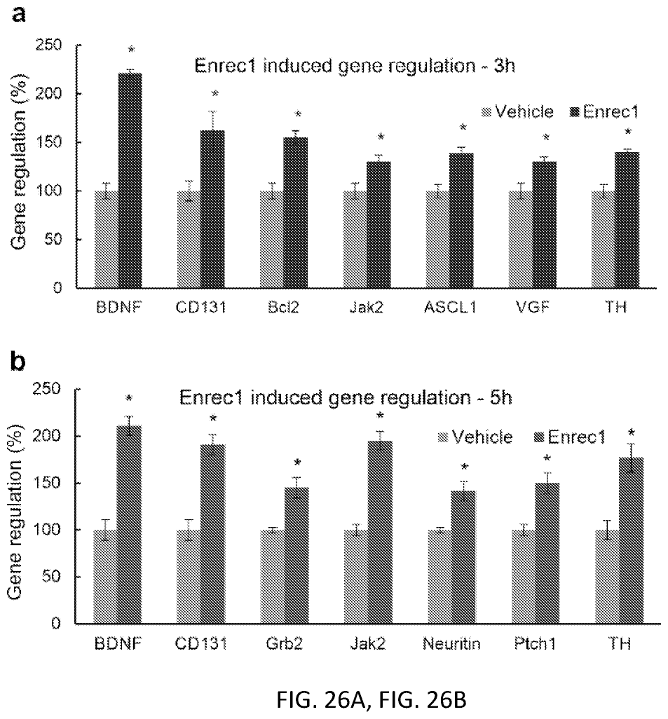

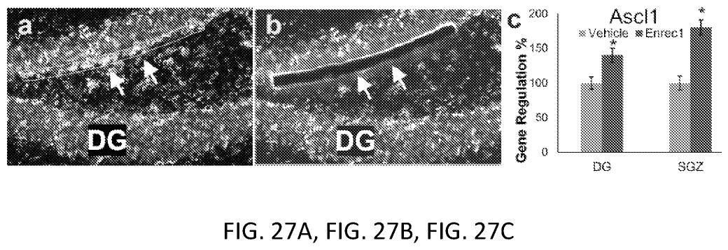

The PC12 line has been used extensively to characterize multiple aspects of EPO signaling. To test the functional activity of EnRec1 NGF-treated PC12 cells (neuronal phenotype) were employed and the regulation of several genes previously implicated in the signaling cascade of EPOR and CEPO were examined. A short list of regulated genes is shown (FIG. 26). The neurotrophic genes included BDNF, VGF and neuritin. These 3 neurotrophins are also induced by EPO and exercise. Interestingly, each of these neurotrophins have been independently shown to produce antidepressant-like effects in rodent models. While EPO and CEPO elevate BDNF by 50%, EnRec1 increased it by 200%, likely due to alteration in angular orientation of receptor binding. CD131 and Ptch have been implicated in CEPO signal transduction and Jak 2 induction suggests that EPOR is activated by EnRec1. CD131 is suggested as the 3rd receptor of a heterotrimeric EPOR complex that is primarily responsible for CEPO possessing neurotrophic but not erythropoietic activity. The proneural transcription factor Ascl1 was transiently regulated, as it increased 30% at 3 h but returned to baseline at 5 h. Ascl1 induction is associated with hippocampal stem activation and is a critical indicator of neurogenic potential. Oscillatory expression of Ascl1 is responsible for maintenance of proliferating neural progenitor cells while sustained elevation results in neuronal fate determination. The increase in tyrosine hydroxylase (TH) could underlie EPOs cognitive enhancing effects that have been reported in clinical studies.

EnRec1 penetrates the brain and regulates Ascl1 in the hippocampus The gene regulation data indicates that EnRec1 activates EPOR and would cross the BBB by receptor-mediated translocation. Nevertheless, several CNS drug targets that produce attractive results in cell lines exhibit poor efficacy in preclinical tests due to their inability to cross the blood brain barrier (BBB). Whether EnRec1 crosses the BBB in mice to elicit functional activity in the CNS was tested. Five, once daily doses of EnRec1 was sufficient to elevate Ascl1 expression in the hippocampus (FIG. 28). The higher induction in the SGZ (FIG. 28c) is of significant interest because recent data has demonstrated that Ascl1 is required and sufficient for neurogenesis, functioning by altering the chromatin landscape. Ascl1 is therefore an ideal target to determine neurogenic potential.

EnRec1 was safe and free of any toxicity based on behavior, body weight, coat condition and grooming.

Experiments Hippocampal (DG and SGZ) gene regulation (BDNF, VGF, neuritin and Ascl1) can be examined with 2 administration regimens (4 and 10 d) to distinguish between faster and slower onset effects that have been observed in the clinic. Both these hippocampal layers have been firmly implicated in antidepressant activity. Behaviorally effective dose can be employed. Methodology previously optimized for LMD can be used. These studies can be conducted in both males and females (C57B16) to test for any gender specific effects. The gene data can be confirmed at the protein level using immunocytochemistry using protocols optimized for cryocut sections.

Alternate plans EnRec1 is currently 85-90% pure and sufficient for animal testing. This level of purity is superior to trophic factors supplied by commercial Life Science vendors that are used in the vast majority of preclinical studies. Although EnRec1 is of bacterial origin it should be noted that 30% of FDA approved protein pharmaceuticals are made in E. coli. If the presence of additional proteins produce any adverse effects it can be purified to homogeneity. In addition to MBP and TEV a 6×Histidine tag (see FIG. 24) was also included into the construct. This was primarily for detection, but can also be used effectively for purification using a conventional high affinity nickel-nitrilotriacetic acid (Ni-NTA) resin.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Organization W H (2019): Mental Health in the Workplace. May ed.
2. Al-Harbi K S (2012): Treatment-resistant depression: therapeutic trends, challenges, and future directions. Patient preference and adherence. 6:369-388.
3. James S L, Abate D, Abate K H, Abay S M, Abbafati C, Abbasi N, et al. (2018): Global, regional, and national incidence, prevalence, and years lived with disability for 354 diseases and injuries for 195 countries and territories, 1990-2017: a systematic analysis for the Global Burden of Disease Study 2017. The Lancet. 392:1789-1858.
4. Miskowiak K W, Vinberg M, Harmer C J, Ehrenreich H, Kessing L V (2012): Erythropoietin: a candidate treatment for mood symptoms and memory dysfunction in depression. Psychopharmacology. 219:687-698.
5. Miskowiak K W, Vinberg M, Christensen E M, Bukh J D, Harmer C J, Ehrenreich H, et al. (2014): Recombinant human erythropoietin for treating treatment-resistant depression: a double-blind, randomized, placebo-controlled phase 2 trial. Neuropsychopharmacology. 39:1399-1408.
6. Miskowiak K W, Vinberg M, Macoveanu J, Ehrenreich H, Koster N, Inkster B, et al. (2015): Effects of Erythropoietin on Hippocampal Volume and Memory in Mood Disorders. Biol Psychiatry. 78:270-277.
7. Shirayama Y, Chen A C-H, Nakagawa S, Russell D S, Duman R S (2002): Brain-Derived Neurotrophic Factor Produces Antidepressant Effects in Behavioral Models of Depression. The Journal of Neuroscience. 22:3251-3261.
8. Girgenti M J, Hunsberger J, Duman C H, Sathyanesan M, Terwilliger R, Newton S S (2009): Erythropoietin Induction by Electroconvulsive Seizure, Gene Regulation, and Antidepressant-Like Behavioral Effects. Biological Psychiatry. 66:267-274.
9. Wilson I A, Jolliffe L K (1999): The structure, organization, activation and plasticity of the erythropoietin receptor. Current opinion in structural biology. 9:696-704.
10. Goldwasser E (1984): Erythropoietin and its mode of action. Blood cells. 10:147-162.
11. Syed R S, Reid S W, Li C, Cheetham J C, Aoki K H, Liu B, et al. (1998): Efficiency of signalling through cytokine receptors depends critically on receptor orientation. Nature. 395:511-516.
12. Jubinsky P T, Krijanovski O I, Nathan D G, Tavernier J, Sieff C A (1997): The β Chain of the Interleukin-3 Receptor Functionally Associates With the Erythropoietin Receptor. Blood. 90:1867-1873.
13. Chen J, Yang Z, Zhang X (2016): Carbamylated Erythropoietin: A Prospective Drug Candidate for Neuroprotection. Biochem Insights. 8:25-29.
14. Leist M, Ghezzi P, Grasso G, Bianchi R, Villa P, Fratelli M, et al. (2004): Derivatives of Erythropoietin That Are Tissue Protective But Not Erythropoietic. Science. 305:239-242.
15. Sampath D, McWhirt J, Sathyanesan M, Newton S S (2020): Carbamoylated erythropoietin produces antidepressant-like effects in male and female mice. Progress in Neuro-Psychopharmacology and Biological Psychiatry. 96:109754.
16. James C. Phillips R B, Wei Wang, James Gumbart, Emad Tajkhorshid, Elizabeth Villa, Christophe Chipot, Robert D. Skeel, Laxmikant Kale, and Klaus Schulten. Scalable molecular dynamics with NAMD. Journal of Computational Chemistry, 26:1781-1802, 2005. abstract, journal.
17. Molecular Operating Environment (MOE) 2013; 08. Chemical Computing Group ULC SSWS, Montreal, QC, Canada: 2018. p. H3A 2R7.
18. Pekas N J, Newton S S (2018): Computational analysis of ligand-receptor interactions in wild-type and mutant erythropoietin complexes. Advances and applications in bioinformatics and chemistry: AABC. 11:1-8.
19. TSUDA E, KAWANISHI G, UEDA M, MASUDA S, SASAKI R (1990): The role of carbohydrate in recombinant human erythropoietin. European Journal of Biochemistry. 188:405-411.
20. Cheung Tung Shing K S, Broughton S E, Nero T L, Gillinder K, Ilsley M D, Ramshaw H, et al. (2018): EPO does not promote interaction between the erythropoietin and beta-common receptors. Scientific Reports. 8:12457.
21. Su, K.-H., Shyue, S.-K., Kou, Y. R., Ching, L.-C., Chiang, A.-N., Yu, Y.-B., . . . Lee, T.-S. (2011). β Common receptor integrates the erythropoietin signaling in activation of endothelial nitric oxide synthase. Journal of Cellular Physiology, 226(12), 3330-3339. doi: 10.1002/jcp.22678

We claim:

1. A polypeptide comprising the sequence APPRLICDSRVLERYLLEAQEAENITTGCAEHCSLNENITVPDTQVNFYAWKRMEVG QQAVEVWQGLALLSEAVLRGQALLVNSSQPWEPLQLHVDQAVSGLRSLTTLLRALG AQKEAISPPDAASAAPLRTITADTFRKLFRVYSNFLRGKLKLYTGEACRTGDR (SEQ ID NO: 1) or a variant thereof.

2. A variant Epo polypeptide comprising three amino acid substitutions at positions 20, 45 and 97 of SEQ ID NO: 3.

3. The variant Epo polypeptide of claim 2, wherein the substitution is a lysine (K) to glutamine (Q) substitution at positions 20, 45 and 97 of SEQ ID NO: 3.

4. The polypeptide or variant Epo polypeptide of any of the preceding claims, further comprising a maltose binding protein sequence.

5. The polypeptide or variant Epo polypeptide of any of the preceding claims, further comprising a histidine tag.

6. The polypeptide or variant Epo polypeptide of any of the preceding claims, further comprising a Factor Xa peptide sequence.

7. A polypeptide comprising the sequence MKIEEGKLVIWINGDKGYNGLAEVGKKFEKDTGIKVTVEHPDKLEEKFPQVAATGD GPDIIFWAHDRFGGYAQSGLLAEITPDKAFQDKLYPFTWDAVRYNGKLIAYPIAVEA LSLIYNKDLLPNPPKTWEEIPALDKELKAKGKSALMFNLQEPYFTWPLIAADGGYAF KYENGKYDIKDVGVDNAGAKAGLTFLVDLIKNKHMNADTDYSIAEAAFNKGETAM TINGPWAWSNIDTSKVNYGVTVLPTFKGQPSKPFVGVLSAGINAASPNKELAKEFLE NYLLTDEGLEAVNKDKPLGAVALKSYEEELAKDPRIAATMENAQKGEIMPNIPQMS AFWYAVRTAVINAASGRQTVDEALKDAQTNSSSNNNNLGIEGRISEFHHH HHHAPPRLICDSRVLERYLLEAQEAENITTGCAEHCSLNENITVPDTQVNFYAWKRM EVGQQAVEVWQGLALLSEAVLRGQALLVNSSQPWEPLQLHVDQAVSGLRSLTTLLR ALGAQKEAISPPDAASAAPLRTITADTFRKLFRVYSNFLRGKLKLYTGEACRTGDR (SEQ ID NO: 2) or variant thereof.

8. The polypeptide of claim 1 or 7 or any of the variant Epo polypeptide of claims 2-6, wherein said polypeptide or variant Epo polypeptide lacks the native carbohydrate moiety in comparison to native human erythropoietin or carbamylated erythropoietin.

9. The polypeptide or variant Epo polypeptide of any of the preceding claims, wherein said polypeptide or variant Epo polypeptide is 40% smaller in comparison to native human erythropoietin.

10. A polynucleotide comprising a nucleic acid capable of encoding the polypeptide or variant Epo polypeptide of any of the preceding claims.

11. A polynucleotide comprising the sequence GCTCCGCCGCGCCTGATCTGTGACTCTCGTGTCCTGGAACGCTATCTGCTG GAAGCGCAGGAAGCCGAAAACATTACCACGGGCTGCGCCGAACATTGTA GCCTGAACGAAAATATCACCGTTCCGGATACGCAGGTCAATTTTTATGCAT GGAAACGTATGGAAGTCGGCCAGCAAGCTGTGGAAGTTTGGCAAGGTCTG GCACTGCTGTCTGAAGCAGTGCTGCGTGGTCAGGCACTGCTGGTTAACAG CTCTCAACCGTGGGAACCGCTGCAGCTGCACGTCGACCAAGCCGTGAGTG GTCTGCGTTCCCTGACCACGCTGCTGCGTGCACTGGGTGCTCAGAAAGAA GCGATTTCACCGCCGGATGCAGCATCGGCAGCTCCGCTGCGTACCATCAC GGCAGACACCTTTCGTAAACTGTTCCGCGTTTACTCCAATTTCCTGCGCGG TAAACTGAAACTGTATACGGGTGAAGCCTGTCGCACGGGTGACCGC (SEQ ID NO. 4) or variant thereof.

12. The polynucleotide of claim 10, wherein said polypeptide or variant Epo polypeptide lacks the native carbohydrate moiety in comparison to native human erythropoietin or carbamylated erythropoietin.

13. The polynucleotide of any one of claims 10-12, wherein said polypeptide or variant Epo polypeptide is 40% smaller in comparison to native human erythropoietin.

14. A vector comprising a polynucleotide of any one of claims 10-13.

15. A cell comprising a polypeptide of claim 1 or 7, a variant Epo polypeptide of claims 2-6, a polynucleotide of any one of claims 10-13 or the vector of claim 14.

16. The cell of claim [00123], wherein said cell is a bacterial cell.

17. A composition comprising a polypeptide of claim 1 or 7, a variant Epo polypeptide of claims 2-6, a polynucleotide of any one of claims 10-13 or the vector of claim 14, or a cell of any one of claims [00123]-[00123].

18. A method of making a variant Epo polypeptide, wherein the variant Epo polypeptide comprises three amino acid substitutions at positions 20, 45 and 97 of SEQ ID NO: 3, the method comprising administering a polynucleotide of any of claims 10-13 or a vector of claim 14 to a cell and culture under conditions that allow for expression of a polypeptide encoded by the polynucleotides.

19. A method of increasing the expression of a neurotrophic gene in a subject, the method comprising:
a. administering a therapeutically effective amount of a polypeptide or variant Epo polypeptide of any of claims 1-9 to the subject;
b. administering a therapeutically effective amount of a polynucleotide of any of claims 10-13 to the subject;
c. administering a therapeutically effective amount of a vector of claim 14 to the subject;
d. administering a therapeutically effective amount of a cell of any of claims 15-16 to the subject; or
e. administering a therapeutically effective amount of a composition of claim 17 to the subject.

20. The method of claim 19, wherein the neurotrophic gene is BDNF, VGF or neuritin.

21. A method of activating EPOR in a subject, the method comprising:
a. administering a therapeutically effective amount of a polypeptide or variant Epo polypeptide of any of claims 1-9 to the subject;
b. administering a therapeutically effective amount of a polynucleotide of any of claims 10-13 to the subject;
c. administering a therapeutically effective amount of a vector of claim 14 to the subject;
d. administering a therapeutically effective amount of a cell of any of claims 15-16 to the subject; or
e. administering a therapeutically effective amount of a composition of claim 17 to the subject.

22. A method of elevating AScll expression in the hippocampus of a subject the method comprising:
a. administering a therapeutically effective amount of a polypeptide or variant Epo polypeptide of any of claims 1-9 to the subject;
b. administering a therapeutically effective amount of a polynucleotide of any of claims 10-13 to the subject;
c. administering a therapeutically effective amount of a vector of claim 14 to the subject;
d. administering a therapeutically effective amount of a cell of any of claims 15-16 to the subject; or
e. administering a therapeutically effective amount of a composition of claim 17 to the subject.

23. A method of treating/ameliorating a symptom of a psychiatric disorder the method comprising:
a. administering a therapeutically effective amount of a polypeptide or variant Epo polypeptide of any of claims 1-9 to the subject;
b. administering a therapeutically effective amount of a polynucleotide of any of claims 10-13 to the subject;
c. administering a therapeutically effective amount of a vector of claim 14 to the subject;
d. administering a therapeutically effective amount of a cell of any of claims 15-16 to the subject; or
e. administering a therapeutically effective amount of a composition of claim 17 to the subject.

24. The method of any of the preceding claims, wherein the subject is anemic.

25. The method of any of the preceding claims, wherein the subject is not anemic.

26. The method of any of claims 18-25, wherein the polypeptide or variant Epo polypeptide; polynucleotide, vector, cell, or composition is administered to the subject by a route selected from the group consisting of orally, buccally, parenterally, nasally, rectally, and topically.

27. The method of any of claims 18-26, further comprising at least one of:
monitoring the subject's red blood cell indices, maintaining the subject's red cell indices at substantially normal levels during treatment, or both.

28. A method of improving cognitive function comprising:
a. administering a therapeutically effective amount of a polypeptide or variant Epo polypeptide of any of claims 1-9 to the subject;
b. administering a therapeutically effective amount of a polynucleotide of any of claims 10-13 to the subject;
c. administering a therapeutically effective amount of a vector of claim 14 to the subject;
d. administering a therapeutically effective amount of a cell of any of claims 15-16 to the subject; or
e. administering a therapeutically effective amount of a composition of claim 17 to the subject.
